(19) United States
(12) Patent Application Publication
(10) Pub. No.: US 2024/0287608 A1
(43) Pub. Date: Aug. 29, 2024

(54) CIRCULATING LEVELS OF ENDOTHELIAL MICRORNA PREDICT NEW-ONSET DIABETES IN COVID-19 AND LONG COVID

(71) Applicant: Albert Einstein College of Medicine, Bronx, NY (US)

(72) Inventor: Gaetano Santulli, New York, NY (US)

(21) Appl. No.: 18/424,483

(22) Filed: Jan. 26, 2024

Related U.S. Application Data
(63) Continuation-in-part of application No. PCT/US23/19592, filed on Apr. 24, 2023.
(60) Provisional application No. 63/335,029, filed on Apr. 26, 2022.

(51) Int. Cl. C12Q 1/6883

(52) U.S. Cl. CPC C12Q 1/6883; C12Q 2600/178

(57) ABSTRACT

Provided herein are compositions and methods for identifying COVID-19 subjects at risk for developing diabetes.

### CROSS-REFERENCE TO RELATED APPLICATIONS

This application is a Continuation-in-Part application of International Application No. PCT/US23/19592, filed on Apr. 24, 2023, which claims the benefit of U.S. Provisional Application No. 63/335,029, filed on Apr. 26, 2022, both of their entire contents are incorporated herein by this reference.

### SEQUENCE LISTING

The instant application contains a Sequence Listing which has been submitted electronically in XML format. Said XML file, created on May 6, 2024, is named “AET-03301.xml” and is 14,714 bytes in size. The sequence listing contained in this .XML file is part of the specification and is hereby incorporated by reference in its entirety.

### BACKGROUND OF THE INVENTION

The coronavirus disease 2019 (COVID-19) pandemic caused by the severe acute respiratory syndrome coronavirus-2 (SARS-COV-2) has led to a global healthcare crisis; ˜80 million Americans and 440 million worldwide have been diagnosed as infected by SARS-COV-2, leading to 1-million and 6-million deaths, respectively.

The relationship between COVID-19 and diabetes is twofold: while it is known that the presence of diabetes and other metabolic alterations poses a considerably high risk to develop a severe COVID-19 (Refs1, 21-23), emerging evidence indicates that patients who survived a SARS-COV-2 infection have an increased risk of new-onset diabetes (Refs 2-6 and 24-49). This aspect is particularly relevant if we consider that chronic manifestations of the disease, including metabolic disturbances, have been reported even months after the initial infection occurred (“Long COVID”) (Refs 6-9 and 50-52).

Despite the relationship between COVID-19 and diabetes, there are no established biomarkers that can be used to determine the risk of developing diabetes in a subject that has or has had COVID-19. There is a huge gap between the demand and the supply of reliable biomarkers that can predict COVID-19 complications, especially considering the unpredictable impact of “long COVID-19” (chronic sequelae of COVID-19).

### SUMMARY OF THE INVENTION

The present invention is based, at least in part, on the discovery that plasma levels of a microRNA (miRNA) are particularly useful in identifying COVID-19 patients who are at risk for developing diabetes. Provided herein are methods of detecting miR-34a; methods of identifying COVID-19 patients who are at risk for developing diabetes; and methods of preventing or treating diabetes in said patients.

### BRIEF DESCRIPTION OF FIGURES

FIG. 1 shows main characteristics of the patient population at hospital admission. Data on quantitative parameters are expressed as mean±standard deviation; data on qualitative characteristics are expressed as absolute numbers and percentage values. BMI: Body mass index; SBP: Systolic blood pressure; DBP: Diastolic blood pressure; IL-6: Interleukin-6; TNFα: Tumor Necrosis Factor α; hs-CRP: high-sensitivity C Reactive Protein; EC-EV miR-34a: level of miR-34a within endothelial cells extracellular vesicles. *P<0.05.

FIG. 2 shows multivariable linear regression analysis assessing the association between miR-34a and new-onset diabetes in COVID-19 patients. BMI: body mass index; EC-EV miR-34a: level of miR-34a within endothelial cells extracellular vesicles.

### DETAILED DESCRIPTION OF THE INVENTION

A significantly augmented risk for newly diagnosed diabetes after SARS-COV-2 infection, independent of steroid use (which was instead linked to transient hyperglycemia), has been recently demonstrated in a large study performed by the CDC (Ref 4). Current COVID-19 guidelines recommend the use of steroids in hospitalized patients up to 10 days or until discharge and should not be prescribed beyond discharge (Ref 101). A retrospective analysis of more than 27-million patients (Center Real-World data) revealed that COVID-19 is associated with an increased risk of diabetes, and that Black, Asian/Pacific Islander, and American Indian/Alaskan Native populations are disproportionately at risk (as described in Ref 85); a significant rise in new-onset type-2 diabetes has been observed among children and adolescents of Alabama during the COVID-19 pandemic (as described in Ref 86); equally important, a meta-analysis has evidenced that the incidence of diabetic ketoacidosis among pediatric patients has significantly increased during COVID-19 pandemic (as described in Ref 87).

Ref 10 demonstrated that survivors of COVID-19 had a 39% increased likelihood of receiving a new diabetes diagnosis within six months following infection compared with noninfected patients; the cohort included 73,435 COVID-19 patients of the Veterans Health Administration, and ˜5 million control subjects who did not have COVID-19 (Ref 10). The same team of researchers lately published another paper evidencing that people with COVID-19 exhibit a markedly augmented risk of new-onset diabetes that increased according to the severity of the infection as proxied by the care setting: non-hospitalized, hospitalized, and admitted to intensive care (Ref 84). Another study conducted in 47,780 hospitalized patients with COVID-19 found that these patients had a ˜50% increased likelihood of developing diabetes ˜20 weeks following discharge compared with matched control patients (Ref 11). In full alignment with these reports, the finding of this disclosure includes the discovery that in a population of more than 200,000 individuals the risk of new-onset diabetes is markedly increased when comparing the years of the pandemic (2020-2022) to the triennium 2017-2019 (as described in Ref 42). These findings provide support for a diabetogenic effect of COVID-19, beyond the well-recognized stress response associated with severe illness.

Quantifying the expression levels of microRNAs (miRNAs) shuttled by extracellular vesicles (EVs) has been shown to be extremely valuable in clinical practice (as described in Refs 88-100). We have recently demonstrated that endothelial EVs (EC-EVs) enriched in miR-24 independentlypredict cerebrovascular events in COVID-19 (Ref 19). Comparing circulating levels of EC-EVs of COVID-19 patients who developed diabetes to COVID-19 patients who did not develop diabetes, we identified miR-34a as one of the top upregulated miRNAs. Therefore, we hypothesized the existence of a significant association between the onset of diabetes and plasma levels of EC-EV miR-34a in patients that have been hospitalized for COVID-19.

The present disclosure provides a method of identifying a COVID-19-positive subject at risk of developing diabetes based on an association between a microRNA in a sample of the COVID-19 subject. In some embodiments, the method comprises determining the level of the microRNA in a sample of the COVID-19-positive subject and comparing the level of the microRNA in the sample to the level of the microRNA in a control sample. In some embodiments, a significantly higher level of the microRNA relative to the control indicates that the COVID-19-positive subject is at risk of developing diabetes. In some embodiments, the sample comprises an endothelial cell extracellular vesicle (EV). In some embodiments, the endothelial cell EV is isolated from the plasma of the COVID-19-positive subject.

In some embodiments, the subject is a mammal. In some embodiments, the COVID-19-positive subject has long COVID.

In some embodiments, the present disclosure demonstrates an association between the plasma level of endothelial extracellular vesicle (EV) miR-34a and the onset of diabetes in COVID-19 patients. Thus, miR-34a level provides an important diagnostic tool in identifying COVID-19 patients who are at risk for developing diabetes. The identified patients can be treated with various therapies of the present disclosure or those known in the art to prevent and/or treat diabetes.

In another aspect, the present disclosure provides a method of preventing or treating diabetes in a COVID-19-positive subject comprising identifying a COVID-19-positive subject at risk for developing diabetes according to the method herein provided and administering to the subject a therapy to prevent or treat diabetes.

### Definitions

The articles “a” and “an” are used herein to refer to one or to more than one (i.e., to at least one) of the grammatical object of the article. By way of example, “an element” means one element or more than one element.

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “extracellular vesicle” or “EV” refers to a lipid-based microparticle or nanoparticle present in a sample (e.g., a biological fluid, body fluid) obtained from a subject. EVs can be referred to as exosomes, microvesicles, and nanovesicles. An EV is generally between about 20 nm to about 90 nm in diameter. EVs are derived from a variety of different mammalian cell types, and can be secreted or shed from such cells.

A “kit” is any manufacture (e.g. a package or container) comprising at least one reagent, e.g. a probe for the miRNA (e.g., a probe for miR-34a), for collecting, isolating, processing, and/or detecting EVs and/or the level of the miRNA (e.g., miR-34a) therein. In certain embodiments, the kit may further comprise a reference standard, e.g., the miRNA in an amount known to identify COVID-19 patients at risk for diabetes (e.g., positive control for a diagnostic assay) or the miRNA in an amount known to identify COVID-19 patients who are not at risk for diabetes (e.g., a negative control for a diagnostic assay). The kit may be promoted, distributed, or sold as a unit for performing the methods encompassed by the present invention. Reagents in the kit may be provided in individual containers or as mixtures of two or more reagents in a single container. In addition, instructional materials which describe the use of the compositions within the kit can be included.

As used herein, “subject” refers to any animal, e.g., a mammal or a human, e.g., healthy or diseased. In preferred embodiments, the subject is a COVID-19-positive subject and/or a subject with long COVID. In some embodiments, a subject has a family history of developing diabetes. In some embodiments, a subject has a lifestyle that may predispose the subject to develop diabetes. In some embodiments, a subject has not experienced one or more of diabetic symptoms. The term “subject” is interchangeable with “patient”. The term “non-human animal” includes all vertebrates, e.g., mammals and non-mammals, such as non-human primates, sheep, dog, cat, cow, chickens, amphibians, reptiles, etc.

### MicroRNA

MicroRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

In some embodiments, a miRNA is associated with a certain disease. In some embodiments, the miRNA is associated with Diffuse Large B-Cell Lymphoma and Pancreatic Ductal Adenocarcinoma. Among the miRNA's related pathways are Toll-like receptor signaling pathway and cell differentiation.

### MicroRNA-34a

In some embodiments, miR-34a is associated with certain diseases including Diffuse Large B-Cell Lymphoma and Pancreatic Ductal Adenocarcinoma. Among its related pathways are Toll-like receptor signaling pathway and Cell differentiation. In some embodiments, the miRNA is miR-34a. In some embodiments, the miRNA is miR-34a as set forth in SEQ ID NO: 1.

### Extracellular Vesicles (EVs)

EVs are cell-derived vesicles with a closed double-layer membrane structure. According to their size and density, EVs mainly include exosomes (30-150 nm), micro vesicles (MVs) (100-1000 nm), and apoptotic bodies or cancer related oncosomes (1-10 μm). EVs exist in virtually all human body fluids including, but not limited to, whole blood, serum, plasma, urine, saliva, etc. EVs are able to carry various molecules, such as proteins, lipids and RNAs on their surface as well as within their lumen. The EV and exosomal surface proteins can mediate organ-specific homing of circulating EVs and exosomes. EVs can comprise biomarkers that can indicate the source from which the EVs are derived. For example, CD31 is a marker often seen on the surface of endothelial cells, and CD31+ EVs can be derived from such cells. As used herein, the term “extracellular vesicles” or “EVs” includes all cell-derived vesicles with a closed double-layer membrane structure derived from multivesicular bodies or from the plasma membrane, including exosomes, microvesicles, and apoptotic bodies.

As demonstrated herein, the contents of EVs (e.g., miR-34a) are able to serve as novel biomarkers for use in the diagnosis, prognosis, and prediction of onset of diabetes in COVID-19 subjects.

In some embodiments, miRNA in a sample is analyzed without partitioning the EVs. In other embodiments, a sample is processed to partition EVs present therein prior to analysis for the level of miRNAs.

### Isolation and Quantification of Extracellular Vesicles

EVs circulate in blood and many other body fluids, with typical concentrations of between 109-1012 vesicles/ml blood. EVs are generally stable and can tolerate multiple cycles of freezing and thawing while preserving structure and molecular contents. Thus, EVs offer a robust source to discover blood-based biomarkers for clinical use. EVs may be directly assayed from the biological samples, such that the level of EVs is determined or the one or more biomarkers in the EV lumen are determined without prior isolation, purification, or concentration of the EVs.

In some embodiments, the method comprises isolating the endothelial cell EVs and/or a CD31-positive EV from the sample before detecting the level of the miRNA (e.g., miR-34a).

In some embodiments, a sample is processed to partition EVs present therein prior to analysis for the presence and/or level of miRNAs, which may increase sensitivity of the detection, especially if the miRNA copy number is less than 20 copies.

Alternatively, in some embodiments, EVs may be purified or concentrated prior to analysis. Analysis of EVs can include quantifying the amount of one or more EV populations in a biological sample. For example, a heterogeneous population of EVs can be quantified, or a homogeneous population of EVs, such as a population of EVs with a particular biomarker profile (i.e., CD31+), or derived from a particular cell type (cell-of-origin specific EVs) can be isolated from a heterogeneous population of EVs and quantified. Analysis of an EV can also include detecting, quantitatively or qualitatively, a particular biomarker profile or a bio-signature, of an EV. An enriched population of EVs can be obtained from a biological sample derived from any cell or cells capable of producing and releasing EVs into the bodily fluid.

Any methods known in the art can be used to partition EVs. For example, kits that allow EV partitioning are available commercially from vendors, e.g., CD31 MicroBeads (130-091-935; Miltenyi Biotec), Mojosort Magnetic beads from Biolegends (Cat #480016); MagCapture Tim 4 Exosome isolation kit from Fujifilm (Cat #293-77601); Dynabeads MyOne TI Carboxylic Acid beads from Thermofisher (Cat #65011); Dynabeads Streptavidin MyOne TI beads from ThermoFisher (Cat #10606D); ExoEasy exosome purification kit from Qiagen (Cat #76064); Plasma/serum exosome purification kit from Nörgen Biotek (Cat #57400); ExoQuick purification reagent from SBI (Cat #EXOQ5™-1). Also known in the art are traditional methods involving sedimentation of small extracellular vesicles (small EVs) using ultracentrifugation (see e.g., Lane et al. (2017) Methods Mol Biol, 1660:111-130), which is incorporated herein by reference. Alternatively, a method comprising Extracellular Vesicle Capture by AnTibody of Choice and Enzymatic Release may be used (see, e.g., Mitchell et al. (2021) J Extracell Vesicles, 10:e12110), which is incorporated herein by reference.

In some embodiments, physical properties of extracellular vesicles may be employed to separate them from a medium or other source material, including separation on the basis of electrical charge (e.g., electrophoretic separation, ion-exchange chromatography), size (e.g., filtration, size-exclusion chromatography, molecular sieving, etc.), density (e.g., regular or gradient centrifugation), Svedberg constant (e.g., sedimentation with or without external force, etc.).

In some embodiments of the present disclosure, EVs can be isolated or captured by using a lipophilic capture agent. In other embodiments of the present disclosure, EVs can be isolated or captured using an antibody having an affinity for the particular molecule on the surface of the extracellular vesicle to bind to the molecule. Antibodies that specifically bind CD31 can be used as marker to capture EVs derived from endothelial cells. In some embodiments the capture antibody is a monoclonal antibody. In some embodiments, the antibody is a polyclonal antibody. In some embodiments, other proteins serve as the markers used to isolate EVs. For example, tetraspanins have been widely used as markers to capture EVs, as they are found in a significant amount of EVs from many different origins. In some embodiments, the capture antibody having an affinity for the proteins associated with an EV may be tethered to a substrate or bound to a second molecule (e.g., magnetic beads) that allows for isolation of the capture antibody-EV complex. For example, in some embodiments of the present disclosure, the capture antibody is tethered to a surface of an array.

In some embodiments, EVs are isolated based on one or more biological properties, and include methods that can employ surface markers (e.g., precipitation, reversible binding to solid phase, fluorescence assisted cell sorting (FACS), separation using magnetic beads or other surfaces, immunoprecipitation or other antibody-mediated separation techniques, etc.). In some embodiments, an antibody or aptamer that specifically binds to CD31 comprises a label such that upon binding to CD31+ EVs, the vesicles are detectable.

Methods for Detecting miRNA

The present application encompasses the correlation of miRNA (e.g., miR-34a) and onset of diabetes. miR-34a is a 64-nucleotide long RNA molecule that are processed into smaller fragment mature RNAs (nucleotide 4-27; and nucleotides 43-64). The mature miR-34a is incorporated into a RNA-induced silencing complex (RISC) that targets mRNAs through imperfect base pairing with the miRNA. The 64-nucleotide long miR-34a precursor or any portion thereof may be used to determine the level of miR-34a in a sample. In some embodiments, the mature miR-34a comprising nucleotide 4-27 is used to determine the level of miR-34a in a sample. In some embodiments, the mature miR-34a comprising nucleotides 43-64 is used to determine the level of miR-34a in a sample. In some embodiments, both mature miR-34a RNA fragments (a fragment comprising nucleotides 4-27 and a fragment comprising nucleotides 43-64) are used to determine the level of miR-34a in a sample.

The sequence of miR-34a is known in the art. SEQ ID NO:1 provides an exemplary nucleic acid sequence of the human miR-34a precursor. Homo sapiens microRNA 34a (MIR34a) (NCBI NR_029610.1) (SEQ ID NO: 1) ggccagctgtgagtgtttctttggcagtgtcttagctggttgttgtgagcaatagtaaggaagcaatcagcaagtatactgccctagaa gtgctgcacgttgtggggccc

*The above sequence includes RNA nucleic acid molecules (e.g., thymidine replaced with uridine), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of SEQ ID NO: 1, or a portion thereof. Also included are complementary sequences. Such nucleic acid molecules can be useful in detecting the presence and/or level of miRNA-34a for the methods described herein.

Oligonucleotide or modified oligonucleotide (e.g. locked nucleic acid) probes that hybridize to a target RNA are contemplated in the present invention. Oligonucleotides can be generated using the target sequence provided herein or known in the art. Methods for designing and producing oligonucleotide probes are well known in the art (see, e.g., Wernersson et al. (2007) Nat Protoc. 2(11):2677-91). In some embodiments, the oligonucleotide probes are used as primers for an amplification assay (e.g., RT-PCR, isothermal amplification, etc.). In some embodiments, the oligonucleotides are detectably labeled (e.g., with a fluorescent peptide or molecule).

Numerous methods for analyzing RNA levels in a sample are known in the art. Examples of assays include but are not limited to multiplex bead-based assays, RNA-seq, next generation sequencing, sequencing, mass spectrometry (e.g., RNA sequencing by LC-MS, cDNA sequencing by LC-MS), microarray, Southern blotting of the cDNA of miRNA, Northern blotting, PCR, RT-PCR, real-time PCR (e.g., TaqMan®), any variation thereof, or any combination of two or more thereof. Accordingly, any methods described herein or those known in the art can be used to collect, analyze, and detect miRNAs from EVs.

### Sequencing

Any of a variety of sequencing reactions known in the art can be used to directly sequence the miRNAs or their complementary DNA (cDNA) counterpart. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159). Notably, mass spectrometry (e.g., LC-MS, LC-MS/MS) may be used to sequence DNA (see Chowdhury and Guengerich (2013) Curr Protoc Nucleic Acid Chem 7: Unit-7.1611) or RNA (Zhang et al. (2019) Nucleic Acids Research 47:e125).

In certain embodiments, detection of miRNA can be accomplished using methods including, but not limited to, sequencing by hybridization (SBH), sequencing by ligation (SBL), quantitative incremental fluorescent nucleotide addition sequencing (QIFNAS), pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al. (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), and the like. High-throughput sequencing methods, e.g., on cyclic array sequencing using platforms such as Roche 454, Illumina Solexa or MiSeq or HiSeq, AB-SOLID, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenom. 1:95-100; and Shi (2001) Clin. Chem. 47:164-172) (see, for example, U.S. Pat. Publ. Nos. 2013/0274117, 2013/0137587, and 2011/0039304).

Next-generation sequencing (NGS) is a technology for determining the sequence of DNA or RNA to study genetic variation associated with diseases or other biological phenomena. Introduced for commercial use in 2005, this method was initially called “massively-parallel sequencing”, because it enabled the sequencing of many DNA strands at the same time, instead of one at a time as with traditional Sanger sequencing by capillary electrophoresis (CE).

Because of the speed, throughput, and accuracy of NGS, NGS enables the interrogation of hundreds to thousands of miRNAs or their cDNA counterparts at one time in multiple samples, as well as discovery and analysis of different types of genomic features in a single sequencing run, from single nucleotide variants (SNVs), to copy number and structural variants, and even RNA fusions. NGS provides the ideal throughput per run, and studies can be performed quickly and cost-effectively. Additional advantages of NGS include lower sample input requirements, higher accuracy, and ability to detect variants at lower allele frequencies than with Sanger sequencing.

Analyzing the whole genome using next-generation sequencing (NGS) delivers a base-by-base view of all genomic alterations, including single nucleotide variants (SNV), insertions and deletions, copy number changes, and structural variations. Paired-end whole-genome sequencing involves sequencing both ends of a DNA fragment, which increases the likelihood of alignment to the reference and facilitates detection of genomic rearrangements, repetitive sequences, and gene fusions.

In some embodiments, the Illumina “Phased Sequencing” platform, which employs a combination of long and short pair-ends, can be used. In other embodiments, the third-generation single-molecule sequencing technologies (e.g., ONT and PacBio) can produce much longer reads of DNA sequences.

In some embodiments, the “Deep Sequencing” or high-coverage version of Illumina NGS can be used. Deep Sequencing refers to sequencing a sample multiple times, sometimes hundreds or even thousands of times. The Deep Sequencing allows detection of miRNA, rare clonal types, cells, or microbes comprising as little as 1% of the original sample. Illumina's NovaSeq performs such whole-genome sequencing efficiently and cost-effectively, and its scalable output generates up to 6 Tb and 20 billion reads in dual flow cell mode with simple streamlined automated workflows.

### RNA-SEQ

RNA-seq allows for high throughput next generation sequencing (NGS), providing both qualitative and quantitative information about the different RNA species present in a given sample. There are many different types of RNA-seq. Direct RNA-seq sequences the RNA in a sample directly. This method avoids the bias introduced by complementary DNA (cDNA) synthesis, polymerase chain reaction (PCR), or adaptor ligation. However, RNA is an unstable molecule, so often RNA-seq workflows begin with conversion of RNA into cDNA.

### Microarray

In certain embodiments, detection of miRNA can be accomplished using microarrays. High-throughput microarrays have been developed to identify and detect miRNAs in a variety of samples, e.g., tissue and cell types (see, e.g., Babak et al., RNA 10:1813 (2004); Calin et al., Proc. Natl. Acad. Sci. USA 101:11755 (2004); Liu et al., Proc. Natl. Acad. Sci. USA 101:9740 (2004); Miska et al., Genome Biol. 5:R68 (2004); Sioud and Røsok, BioTechniques 37:574 (2004); Krichevsky et al., RNA 9:1274 (2003)). The use of microarrays has several advantages for detection of miRNA expression, including the ability to determine the presence and/or level of multiple miRNAs in the same sample at a single time point, a need for only small amounts of RNA, and the potential to simultaneously identify the expression of both precursor and mature miRNA molecules.

In some embodiments, covalent attachment of fluorophores can be used to directly label miRNA molecules for use in microarray analyses (see, e.g., Babak et al., RNA 10:1813 (2004); MICROMAX ASAP miRNA Chemical Labeling Kit, Perkin Elmer, Shelton, CT; Label IT® μArray Labeling Kit, Mirus Bio Corp., Madison, WI). In other embodiments, random primed-reverse transcription of miRNA molecules can be used to produce labeled cDNA molecules for use in microarray analyses (see, e.g., Sioud and Røsok, BioTechniques 37:574 (2004); Liu et al., Proc. Natl. Acad. Sci. USA 101:9740 (2004)). In some embodiments, the methods herein provided comprise reverse transcribing the miRNA into a cDNA before detecting the level of the miRNA. In some embodiments, the methods herein provided comprise reverse transcribing the miR-34a into a cDNA before detecting the level of the miR-34a.

### Significant Level

The “level” or “amount” of a biomarker (e.g., miRNAs) in a subject is “significantly” higher or lower than the level of a biomarker in a control (e.g., normal sample), if the amount of the biomarker is greater or less, respectively, than the level in a control by an amount greater than the standard error of the assay employed to assess amount.

In some embodiments, the amount or level of a biomarker in a subject can be considered “significantly” higher or lower than the normal and/or control amount if the amount is at least or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 1%-100%, higher or lower, respectively, than the normal and/or control amount of the biomarker. Such significant modulation values can be applied to any metric described herein, such as the level of miRNA.

### Control

A control refers to any reference standard suitable to provide a comparison to the level of products in the test sample. In certain embodiments, the control comprises obtaining a control sample from which the level of the miRNA is detected and compared to the same from the test sample. Such a control sample may comprise any suitable sample, including but not limited to a sample from a control healthy patient (can be stored sample or previous sample measurement) with a known outcome. In some embodiments, the control may comprise a reference standard expression product (e.g., miRNA) level from any suitable source, including but not limited to an expression product level range from normal tissue (or other previously analyzed control sample), a previously determined expression product level range within a test sample from a group of patients, or a set of patients with a certain outcome (for example, a healthy subject, a COVID-19-negative subject, a subject without diabetes, a COVID-19-positive subject without diabetes, etc.) or receiving a certain treatment (for example, COVID-19 therapy). In some embodiments, the control comprises samples drawn or collected longitudinally at different times, to evaluate a change in the level of miRNA over time. It will be understood by those of skill in the art that such control samples and reference standard expression product levels can be used in combination as controls in the methods of the present disclosure.

In some embodiments, the amount of miRNA may be determined within a sample relative to, or as a ratio of, the amount of another housekeeping miRNA in the same sample. In some embodiments, the control comprises a ratio transformation of expression product levels, including but not limited to determining a ratio of product levels of two miRNAs in the test sample and comparing it to any suitable ratio of the same in a reference standard; determining product levels of the two or more miRNAs in the test sample and determining a difference in product levels in any suitable control; and determining product levels of the two or more miRNAs in the test sample, normalizing their level to the level of housekeeping gene products in the test sample, and comparing to any suitable control. In preferred embodiments, the control comprises a control sample which is of the same lineage and/or type as the test sample. In other embodiments, instead of normalizing relative to the level of a housekeeping miRNA, the miRNA level may be normalized relative to any protein level (e.g., CD31), nucleic acid level, lipid level, or others within the sample.

In other embodiments, the control may comprise product levels grouped as percentiles within or based on a set of patient samples, such as all COVID-19 patients. In some embodiments, a control product level is established wherein higher or lower levels of product relative to, for instance, a particular percentile, are used as the basis for predicting outcome. In other preferred embodiments, a control product level is established using product levels from COVID-19 control patients with a known outcome, and the product levels from the test sample are compared to the control product level as the basis for predicting outcome. As demonstrated by the data provided herein, the methods of the present disclosure are not limited to use of a specific cut-point in comparing the level of product in the test sample to the control.

In some embodiments, the control is a pre-determined level of the miRNA. In some embodiments, a pre-determined marker amount (e.g., pre-determined level of the miRNA) can be any suitable standard. For example, the pre-determined marker amount can be obtained from the same or a different human for whom a patient selection is being assessed. In some embodiments, the pre-determined marker amount can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

Accordingly, in some embodiments, a control comprises a sample (e.g., a biological sample or a derivative thereof, a body fluid or a derivative thereof, e.g., plasma or a derivative thereof) from (a) a normal healthy person, a COVID-19-negative subject, a subject without diabetes, or a COVID-19-positive subject without diabetes; or (b) a portion or all of pooled samples from one or more subjects of (a).

In other embodiments, a control comprises a sample (e.g., a biological sample or a derivative thereof, e.g., body fluid or a derivative thereof, e.g., plasma or a derivative thereof) from a patient who is being evaluated (e.g., diagnosis or prognosis). For example, the control sample may comprise (i) a historical sample of the patient, or (ii) the sample obtained from the patient in longitudinal studies, e.g., pre-therapy or post-therapy (e.g., COVID-19 therapy or diabetes). The use of such control allows comparison of a biomarker present in the same patient over time (e.g., during the progression of diabetes).

### Diabetes

Diabetes, also known as diabetes mellitus, is a group of common endocrine diseases characterized by sustained high blood sugar levels. Diabetes is due to either the pancreas not producing enough insulin, or the cells of the body not responding properly to the insulin produced. Diabetes, if left untreated, leads to many health complications. Untreated or poorly treated diabetes accounts for approximately 1.5 million deaths per year.

The classic symptoms of untreated diabetes are unintended weight loss, polyuria (increased urination), polydipsia (increased thirst), and polyphagia (increased hunger). Symptoms may develop rapidly (weeks or months) in type 1 diabetes, while they usually develop much more slowly and may be subtle or absent in type 2 diabetes.

Several other signs and symptoms can mark the onset of diabetes although they are not specific to the disease. In addition to the known symptoms listed above, they include blurred vision, headache, fatigue, slow healing of cuts, and itchy skin. Prolonged high blood glucose can cause glucose absorption in the lens of the eye, which leads to changes in its shape, resulting in vision changes. Long-term vision loss can also be caused by diabetic retinopathy. A number of skin rashes that can occur in diabetes are collectively known as diabetic dermadromes.

Diabetes mellitus is classified into six categories: type 1 diabetes, type 2 diabetes, hybrid forms of diabetes, hyperglycemia first detected during pregnancy, “unclassified diabetes”, and “other specific types”. “Hybrid forms of diabetes” include slowly evolving, immune-mediated diabetes of adults and ketosis-prone type 2 diabetes. “Hyperglycemia first detected during pregnancy” includes gestational diabetes mellitus and diabetes mellitus in pregnancy (type 1 or type 2 diabetes first diagnosed during pregnancy). The “other specific types” are a collection of a few dozen individual causes. Diabetes is a more variable disease than once thought and people may have combinations of forms.

### Type 1

Type 1 diabetes is characterized by loss of the insulin-producing beta cells of the pancreatic islets, leading to insulin deficiency. This type can be further classified as immune-mediated or idiopathic. The majority of type 1 diabetes is of an immune-mediated nature, in which a T cell-mediated autoimmune attack leads to the loss of beta cells and thus insulin. It causes approximately 10% of diabetes mellitus cases in North America and Europe. Most affected people are otherwise healthy and of a healthy weight when onset occurs. Sensitivity and responsiveness to insulin are usually normal, especially in the early stages. Although it has been called “juvenile diabetes” due to the frequent onset in children, the majority of individuals living with type 1 diabetes are now adults.

“Brittle” diabetes, also known as unstable diabetes or labile diabetes, is a term that was traditionally used to describe the dramatic and recurrent swings in glucose levels, often occurring for no apparent reason in insulin-dependent diabetes. This term, however, has no biologic basis and should not be used. Still, type 1 diabetes can be accompanied by irregular and unpredictable high blood sugar levels, and the potential for diabetic ketoacidosis or serious low blood sugar levels. Other complications include an impaired counter regulatory response to low blood sugar, infection, gastroparesis (which leads to erratic absorption of dietary carbohydrates), and endocrinopathies (e.g., Addison's disease). These phenomena are believed to occur no more frequently than in 1% to 2% of persons with type 1 diabetes.

Type 1 diabetes is partly inherited, with multiple genes, including certain HLA genotypes, known to influence the risk of diabetes. In genetically susceptible people, the onset of diabetes can be triggered by one or more environmental factors.

Type 1 diabetes can occur at any age, and a significant proportion is diagnosed during adulthood. Latent autoimmune diabetes of adults (LADA) is the diagnostic term applied when type 1 diabetes develops in adults; it has a slower onset than the same condition in children.

### Type 2

Type 2 diabetes is characterized by insulin resistance, which may be combined with relatively reduced insulin secretion. The defective responsiveness of body tissues to insulin is believed to involve the insulin receptor. However, the specific defects are not known. Diabetes mellitus cases due to a known defect are classified separately. Type 2 diabetes is the most common type of diabetes mellitus accounting for 95% of diabetes. Many people with type 2 diabetes have evidence of prediabetes (impaired fasting glucose and/or impaired glucose tolerance) before meeting the criteria for type 2 diabetes. The progression of prediabetes to overt type 2 diabetes can be slowed or reversed by lifestyle changes or medications that improve insulin sensitivity or reduce the liver's glucose production.

Type 2 diabetes is primarily due to lifestyle factors and genetics. A number of lifestyle factors are known to be important to the development of type 2 diabetes, including obesity (defined by a body mass index of greater than 30), lack of physical activity, poor diet, stress, and urbanization. Excess body fat is associated with 30% of cases in people of Chinese and Japanese descent, 60-80% of cases in those of European and African descent, and 100% of Pima Indians and Pacific Islanders. Even those who are not obese may have a high waist-hip ratio.

Dietary factors such as sugar-sweetened drinks are associated with an increased risk. The type of fats in the diet is also important, with saturated fat and trans fats increasing the risk and polyunsaturated and monounsaturated fat decreasing the risk. Eating white rice excessively may increase the risk of diabetes, especially in Chinese and Japanese people. Lack of physical activity may increase the risk of diabetes in some people.

Adverse childhood experiences, including abuse, neglect, and household difficulties, increase the likelihood of type 2 diabetes later in life by 32%, with neglect having the strongest effect.

Antipsychotic medication side effects (specifically metabolic abnormalities, dyslipidemia and weight gain) and unhealthy lifestyles (including poor diet and decreased physical activity), are potential risk factors.

### Other Types

Gestational diabetes resembles type 2 diabetes in several respects, involving a combination of relatively inadequate insulin secretion and responsiveness. It occurs in about 2-10% of all pregnancies and may improve or disappear after delivery. It is most often diagnosed in the second or third trimester because of the increase in insulin-antagonist hormone levels that occurs at this time. However, after pregnancy approximately 5-10% of women with gestational diabetes are found to have another form of diabetes, most commonly type 2. Gestational diabetes is fully treatable, but requires careful medical supervision throughout the pregnancy. Management may include dietary changes, blood glucose monitoring, and in some cases, insulin may be required.

Maturity onset diabetes of the young (MODY) is a rare autosomal dominant inherited form of diabetes, due to one of several single-gene mutations causing defects in insulin production. It is significantly less common than the three main types, constituting 1-2% of all cases. The name of this disease refers to early hypotheses as to its nature. Being due to a defective gene, this disease varies in age at presentation and in severity according to the specific gene defect; thus, there are at least 13 subtypes of MODY. People with MODY often can control it without using insulin.

Some cases of diabetes are caused by the body's tissue receptors not responding to insulin (even when insulin levels are normal, which is what separates it from type 2 diabetes); this form is very uncommon. Genetic mutations (autosomal or mitochondrial) can lead to defects in beta cell function. Abnormal insulin action may also have been genetically determined in some cases. Any disease that causes extensive damage to the pancreas may lead to diabetes (for example, chronic pancreatitis and cystic fibrosis). Diseases associated with excessive secretion of insulin-antagonistic hormones can cause diabetes (which is typically resolved once the hormone excess is removed). Many drugs impair insulin secretion and some toxins damage pancreatic beta cells, whereas others increase insulin resistance (especially glucocorticoids which can provoke steroid diabetes). Yet another form of diabetes that people may develop is double diabetes. This is when a type 1 diabetic becomes insulin resistant, the hallmark for type 2 diabetes or has a family history for type 2 diabetes.

### Diagnostic Methods

The present disclosure provides, in part, methods, systems, and compositions for accurately classifying whether a biological sample comprises miRNA and/or whether the levels of miRNA are modulated (e.g., upregulated or downregulated), thereby indicative of the state of a disorder of interest, such as onset of diabetes in COVID-19 patients. In some embodiments, the present invention is useful for classifying a sample (e.g., from a subject) as associated with or at risk for developing diabetes or a symptom thereof, using a statistical algorithm and/or empirical data (e.g., the presence, absence, or level miRNA).

An exemplary method for detecting the level of miRNA involves obtaining a biological sample (e.g., a biological sample or a derivative thereof, a body fluid or a derivative thereof, e.g., plasma or a derivative thereof) from a test subject and detecting miRNA in the sample using methods described herein or those known in the art.

In certain instances, the statistical algorithm is a single learning statistical classifier system. For example, a single learning statistical classifier system can be used to classify a sample as a at-risk diabetes sample or a diabetes sample based upon a prediction or probability value and the presence or level of miRNA. The use of a single learning statistical classifier system typically classifies the sample as a at-risk diabetes sample or a diabetes sample with a sensitivity, specificity, positive predictive value, negative predictive value, and/or overall accuracy of at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present disclosure further comprises sending the sample classification results to a clinician (a non-specialist, e.g., primary care physician; and/or a specialist).

In some embodiments, the method of the present disclosure further provides a diagnosis in the form of a probability that the individual has diabetes or at risk of having diabetes. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having diabetes or at risk of having diabetes. In yet another embodiment, the method of the present invention further provides a prognosis of diabetes in the individual (e.g., COVID-19 patient). In some instances, the method of classifying a sample as a at-risk diabetes sample or a diabetes sample may be further based on the symptoms (e.g., clinical factors) of the individual from which the sample is obtained. In some instances, the method of classifying a sample as a diabetes sample or at-risk diabetes sample may be further based on a family history of having diabetes or being at risk of diabetes, irrespective of the symptoms. In some embodiments, the diagnosis of an individual as having diabetes or being at risk of diabetes is followed by administering to the individual a therapeutically effective amount of a therapy (e.g., a diabetes therapy, a COVID-19 therapy). In some embodiments, the diagnosis of an individual as having diabetes is followed by treating the individual with a diabetes therapy to prevent the onset of diabetes.

In some embodiments, the methods further comprise analyzing the control sample to detect miRNA, such that the presence and/or the level of said miRNA is detected in the control biological sample, and comparing the presence or the level of miRNA in the control sample with the presence or the level of miRNA in the test sample.

### Samples

A sample used for the methods and compositions of the present disclosure may comprise any biological sample or a derivative thereof. In some embodiments, a sample comprises a tissue or a cell. In some embodiments, a sample comprises a body fluid or a derivative thereof. The term “body fluid” refers to fluids that are excreted or secreted from the body as well as fluid that are normally not (e.g., blood and blood plasma saliva, serum, tears, urine, sweat). In some embodiments, a sample comprises plasma or a derivative thereof. In preferred embodiments, a sample comprises EVs. In some embodiments, the EVs comprise CD31 protein. In some embodiments, the EVs are endothelial cell EVs.

In certain instances, the method encompassed by the present invention further comprises obtaining the sample from the individual prior to detecting or determining the presence or level of at least one marker in the sample.

A sample can be analyzed as a crude unpartitioned sample, or partitioned to EVs and supernatant. For miRNA that is particularly low in abundance, partitioning a sample to an EV fraction may enhance sensitivity.

In some embodiments, the method comprises fractionating EVs from the sample before detecting the level of the miRNA. In some embodiments, the method comprises fractionating EVs from the sample before detecting the level of the miR-34a. The sample or the partitioned EVs may be then extracted for nucleic acids, optionally followed by size separation for focus on microRNA level evaluations. The microRNA fraction (exosomal or non-exosomal) can be analyzed by next generation sequencing, PCR, or analogous strategies described herein or known in the art.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of RNase inhibitor, addition of denaturants, desalting of samples, concentration of sample RNA, extraction and purification of miRNA.

In some embodiments, the level of miRNA measurement(s) in a sample from a subject is compared to a control biological sample. In other embodiments, the level of miRNA measurement(s) in a sample from a subject is compared to a predetermined control (standard) sample. The sample from the subject may be a COVID-19-positive subject. The control sample can be from the same subject or from a different subject. The control sample can be from a normal, non-diseased subject. The control sample can be a combination of samples from several different subjects. In some embodiments, the level of miRNA from a subject is compared to a pre-determined level. This pre-determined level may be obtained from normal samples.

As described herein, a “pre-determined” biomarker amount measurement(s) may be a biomarker amount measurement(s) used to, by way of example only, evaluate a subject that may be selected for treatment, evaluate a response to a diabetes therapy, and/or evaluate a response to a diabetes therapy. A pre-determined biomarker amount measurement(s) may be determined in populations of patients with or without COVID-19. The pre-determined biomarker amount measurement(s) can be a single number, equally applicable to every patient, or the pre-determined biomarker amount measurement(s) can vary according to specific subpopulations of patients. Age, weight, height, and other factors of a subject may affect the pre-determined biomarker amount measurement(s) of the individual. Furthermore, the pre-determined biomarker amount can be determined for each subject individually. In some embodiments, the amounts determined and/or compared in a method described herein are based on absolute measurements.

In some embodiments, the amounts determined and/or compared in a method described herein are based on relative measurements, such as ratios (e.g., biomarker (e.g., miRNA) level before a treatment vs. after a treatment, such biomarker measurements relative to a spiked or man-made control, such biomarker measurements relative to the expression of a housekeeping gene/miRNA, and the like). For example, the relative analysis can be based on the ratio of pre-treatment biomarker measurement as compared to post-treatment biomarker measurement. Pre-treatment biomarker measurement can be made at any time prior to initiation of a diabetes therapy and/or a COVID-19 therapy. Post-treatment biomarker measurement can be made at any time after initiation of a diabetes therapy and/or a COVID-19 therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of a diabetes therapy and/or a COVID-19 therapy, and even longer toward indefinitely for continued monitoring. Treatment can comprise one or more diabetes therapies and/or COVID-19 therapy therapies.

The pre-determined biomarker amount measurement(s) can be any suitable standard. For example, the pre-determined biomarker amount measurement(s) can be obtained from the same or a different human for whom a patient selection is being assessed. In some embodiments, the pre-determined biomarker amount measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present disclosure, the change of biomarker (e.g., miRNA) amount measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0-fold or greater, or any range in between, inclusive. In some embodiments, the change of biomarker amount measurement(s) from the pre-determined level is a significant level (see above). Such cutoff values apply equally when the measurement is based on relative changes, such as based on the ratio of pre-treatment biomarker measurement as compared to post-treatment biomarker measurement.

Method of Prevention and/or Treatment

The methods of the present disclosure (e.g., detecting miR-34a level, identifying or diagnosing a subject at risk for a diabetes event) may further comprise recommending, prescribing, and/or administering a therapy to prevent or treat diabetes to the COVID-19-positive subject determined to be at risk for developing diabetes.

The term “preventing” is art-recognized, and when used in relation to a condition or a disease, e.g., diabetes, is well understood in the art, and includes administration of a treatment, e.g., a composition which reduces the frequency of, or delays the onset of, symptoms of a medical condition in a subject relative to a subject which does not receive the treatment. Thus, prevention of diabetes includes, for example, reducing one or more symptoms of diabetes in a population of patients receiving a prophylactic treatment relative to an untreated control population, and/or delaying the appearance of one or more symptoms of diabetes in a treated population versus an untreated control population, e.g., by a statistically and/or clinically significant amount.

A “therapeutically effective amount” of a compound is an amount capable of producing a medically desirable result in a treated patient, e.g., prevent diabetes, reduce or alleviate any symptom associated with diabetes, with an acceptable benefit: risk ratio, preferably in a human and/or non-human mammal.

The term “treating” includes prophylactic and/or therapeutic treatments. The term “prophylactic or therapeutic” treatment is art-recognized and includes administration to the subject of one or more of a therapy described herein or those known in the art (e.g., a therapy that prevents or treats diabetes, a therapy that prevents or treats COVID-19, etc.). If it is administered prior to clinical manifestation of the unwanted condition (e.g., disease or other unwanted state of the subject), then the treatment is prophylactic (i.e., it protects the subject against developing the unwanted condition); whereas, if it is administered after manifestation of the unwanted condition, the treatment is therapeutic (i.e., it is intended to diminish, ameliorate, or stabilize the existing unwanted condition or side effects thereof).

In some aspects, provided herein are methods for preventing and/or treating COVID-19 (i.e., a SARS-COV-2 infection).

The methods described herein may be used to treat any subject in need thereof. As used herein, a “subject in need thereof” includes any subject who has COVID-19, who has had COVID-19 and/or who is predisposed to COVID-19. For example, in some embodiments, the subject has a COVID-19. In some embodiments, the subject has undergone treatments for COVID-19. In some embodiments, the subject is predisposed to COVID-19 due to age, or having a compromised immune system or other serious underlying medical conditions that predisposes the subject to COVID-19. In preferred embodiments, the subject has been tested positive for COVID-19.

The therapies (e.g., diabetes therapy, COVID-19 therapy) described herein or those known in the art, or pharmaceutical compositions comprising same may be delivered by any suitable route of administration, including orally and parenterally. In certain embodiments the pharmaceutical compositions are delivered generally (e.g., via oral or parenteral administration). In certain embodiments, the pharmaceutical compositions are administered by subcutaneous injection.

The dosage of the subject agent (e.g., diabetes therapy, COVID-19 therapy, or pharmaceutical composition comprising same) may be determined by reference to the plasma concentrations of the agent. For example, the maximum plasma concentration (Cmax) and the area under the plasma concentration-time curve from time 0 to infinity (AUC (0-4)) may be used. Dosages include those that produce the above values for Cmax and AUC (0-4) and other dosages resulting in larger or smaller values for those parameters.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

A pharmaceutical dosage unit may be applied to a subject in a single volume, e.g., a single shot, or may be applied in 2, 3, 4, 5 or more separate volumes or shots that are applied at different locations of the body, for instance in the right and the left limb. Reasons for applying a single pharmaceutical dosage unit in separate volumes may be multiples, such as avoid negative side effects. It is to be understood herein that the separate volumes of a pharmaceutical dosage may differ in composition, i.e., may comprise different kinds or composition of active ingredients and/or adjuvants.

A pharmaceutical dosage unit may be an effective amount or part of an effective amount. An “effective amount” is to be understood herein as an amount or dose of active ingredients required to prevent and/or reduce the symptoms of a disease (e.g., COVID-19, diabetes) relative to an untreated patient. The effective amount of active compound(s) used to practice the present invention for preventive and/or therapeutic treatment of COVID-19 or diabetes varies depending upon the manner of administration, the age, body weight, and general health of the subject. Ultimately, the attending physician or veterinarian will decide the appropriate amount and dosage regimen. Such amount is referred to as an “effective” amount.

### Exemplary Diabetes Therapies

Therapies that prevent or treat diabetes are art-recognized. Provided herein are exemplary therapies.

Drugs used in diabetes treat diabetes mellitus by altering the glucose level in the blood. With the exceptions of insulin, most GLP receptor agonists (liraglutide, exenatide, and others), and pramlintide, all are administered orally and are thus also called oral hypoglycemic agents or oral antihyperglycemic agents. There are different classes of anti-diabetic drugs, and their selection depends on the nature of the diabetes, age and situation of the person, as well as other factors.

Diabetes mellitus type 1 is a disease caused by the lack of insulin. Insulin must be used in type 1, which must be injected.

Diabetes mellitus type 2 is a disease of insulin resistance by cells. Type 2 diabetes mellitus is the most common type of diabetes. Treatments include agents that (1) increase the amount of insulin secreted by the pancreas, (2) increase the sensitivity of target organs to insulin, (3) decrease the rate at which glucose is absorbed from the gastrointestinal tract, and (4) increase loss of glucose through urination.

Several groups of drugs, mostly given by mouth, are effective in type 2, often in combination. The therapeutic combination in type 2 may include insulin, not necessarily because oral agents have failed completely, but in search of a desired combination of effects. The great advantage of injected insulin in type 2 is that a patient can adjust the dose, or even take additional doses, when blood glucose levels are measured by the patient, usually with a simple meter, as needed by the measured amount of sugar in the blood.

### Insulin

Insulin is usually given subcutaneously, either by injections or by an insulin pump. In acute care settings, insulin may also be given intravenously. Insulins are typically characterized by the rate at which they are metabolized by the body, yielding different peak times and durations of action. Faster-acting insulins peak quickly and are subsequently metabolized while longer-acting insulins tend to have extended peak times and remain active in the body for more significant periods.

Examples of rapid-acting insulins (peak at ˜1 hour) are:
Insulin lispro (Humalog)
Insulin aspart (Novolog)
Insulin glulisine (Apidra)
   Examples of short-acting insulins (peak 2-4 hours) are:
Regular insulin (Humulin R, Novolin R)
Prompt insulin zinc (Semilente)
   Examples of intermediate-acting insulins (peak 4-10 hours) are:
Isophane insulin, neutral protamine Hagedorn (NPH) (Humulin N, Novolin N)
Insulin zinc (Lente)
   Examples of long-acting insulins (duration 24 hours, often without peak) are:
Extended insulin zinc insulin (Ultralente)
Insulin glargine (Lantus)
Insulin detemir (Levemir)
Insulin degludec (Tresiba)
   Insulin degludec is sometimes classed separately as an “ultra-long” acting insulin due to its duration of action of about 42 hours, compared with 24 hours for most other long-acting insulin preparations.

A systematic review of studies comparing insulin detemir, insulin glargine, insulin degludec and NPH insulin did not show any clear benefits or serious adverse effects for any particular form of insulin for nocturnal hypoglycemia, severe hypoglycemia, glycated hemoglobin A1c, non-fatal myocardial infarction/stroke, health-related quality of life or all-cause mortality. The same review did not find any differences in effects of using these insulin analogues between adults and children.

### Sensitizers

Insulin sensitizers address the core problem in type 2 diabetes-insulin resistance.

### Biguanides

Biguanides reduce hepatic glucose output and increase uptake of glucose by the periphery, including skeletal muscle. Although it must be used with caution in patients with impaired liver or kidney function, metformin, a biguanide, has become the most commonly used agent for type 2 diabetes in children and teenagers. Among common diabetic drugs, metformin is the only widely used oral drug that does not cause weight gain.

Typical reduction in glycated hemoglobin (A1C) values for metformin is 1.5-2.0%

Phenformin (DBI) was used from 1960s through 1980s, but was withdrawn due to lactic acidosis risk.

Buformin also was withdrawn due to lactic acidosis risk.

Metformin (Glucophage) may be the best choice for patients who also have heart failure, but it should be temporarily discontinued before any radiographic procedure involving intravenous iodinated contrast, as patients are at an increased risk of lactic acidosis. Metformin is usually the first-line medication used for treatment of type 2 diabetes. In general, it is prescribed at initial diagnosis in conjunction with exercise and weight loss, as opposed to in the past, where it was prescribed after diet and exercise had failed. There is an immediate-release as well as an extended-release formulation, typically reserved for patients experiencing gastrointestinal side-effects. It is also available in combination with other oral diabetic medications.

### Thiazolidinediones

Thiazolidinediones (TZDs), also known as “glitazones,” bind to PPARγ, peroxisome proliferator activated receptor γ, a type of nuclear regulatory protein involved in transcription of genes regulating glucose and fat metabolism. These PPARs act on peroxisome proliferator responsive elements (PPRE). The PPREs influence insulin-sensitive genes, which enhance production of mRNAs of insulin-dependent enzymes. The final result is better use of glucose by the cells. These drugs also enhance PPAR-α activity and hence lead to a rise in HDL and some larger components of LDL.

Typical reductions in glycated hemoglobin (A1C) values are 1.5-2.0%. Some examples are:
Rosiglitazone (Avandia): the European Medicines Agency recommended in September 2010 that it be suspended from the EU market due to elevated cardiovascular risks.
Pioglitazone (Actos): remains on the market but has also been associated with increased cardiovascular risks.
Troglitazone (Rezulin): used in 1990s, withdrawn due to hepatitis and liver damage risk.
   Multiple retrospective studies have resulted in a concern about rosiglitazone's safety, although it is established that the group, as a whole, has beneficial effects on diabetes. The greatest concern is an increase in the number of severe cardiac events in patients taking it. The ADOPT study showed that initial therapy with drugs of this type may prevent the progression of disease, as did the DREAM trial. The American Association of Clinical Endocrinologists (AACE), which provides clinical practice guidelines for management of diabetes, retains thiazolidinediones as recommended first, second, or third line agents for type 2 diabetes mellitus, as of their 2019 executive summary, over sulfonylureas and α-glucosidase inhibitors. However, they are less preferred than GLP-1 agonists or SGLT2 inhibitors, especially in patients with cardiovascular disease (which liraglutide, empagliflozin, and canagliflozin are all FDA approved to treat).

### Lyn Kinase Activators

The LYN kinase activator tolimidone has been reported to potentiate insulin signaling in a manner that is distinct from the glitazones. The compound has demonstrated positive results in a Phase 2a clinical study involving 130 diabetic subjects.

### Secretagogues

Secretagogues are drugs that increase output from a gland, in the case of insulin from the pancreas.

### Sulfonylureas

Sulfonylureas were the first widely used oral anti-hyperglycemic medications. They are insulin secretagogues, triggering insulin release by inhibiting the KATP channel of the pancreatic beta cells. Eight types of these pills have been marketed in North America, but not all remain available. The “second-generation” drugs are now more commonly used. They are more effective than first-generation drugs and have fewer side-effects. All may cause weight gain.

Current clinical practice guidelines from the AACE rate sulfonylureas (as well as glinides) below all other classes of antidiabetic drugs in terms of suggested use as first, second, or third line agents—this includes bromocriptine, the bile acid sequestrant colesevelam, α-glucosidase inhibitors, TZDs (glitazones), and DPP-4 inhibitors (gliptins). The low cost of most sulfonylureas, however, especially when considering their significant efficacy in blood glucose reduction, tends to keep them as a more feasible option in many patients-neither SGLT2 inhibitors nor GLP-1 agonists, the classes most favored by the AACE guidelines after metformin, are currently available as generics.

Sulfonylureas bind strongly to plasma proteins. Sulfonylureas are useful only in type 2 diabetes, as they work by stimulating endogenous release of insulin. They work best with patients over 40 years old who have had diabetes mellitus for under ten years. They cannot be used with type 1 diabetes, or diabetes of pregnancy. They can be safely used with metformin or glitazones. The primary side-effect is hypoglycemia, which appears to happen more commonly with sulfonylureas than with other treatments.

### First-Generation Agents

tolbutamide
acetohexamide
tolazamide
chlorpropamide

### Second-Generation Agents

glipizide
glyburide or glibenclamide
glimepiride
gliclazide
glyclopyramide
gliquidone

### Nonsulfonylurea Secretagogues

Meglitinides help the pancreas produce insulin and are often called “short-acting secretagogues.” They act on the same potassium channels as sulfonylureas, but at a different binding site. By closing the potassium channels of the pancreatic beta cells, they open the calcium channels, thereby enhancing insulin secretion.

They are taken with or shortly before meals to boost the insulin response to each meal. If a meal is skipped, the medication is also skipped.

Typical reductions in glycated hemoglobin (A1C) values are 0.5-1.0%.
repaglinide
nateglinide
   Adverse reactions include weight gain and hypoglycemia.

### Alpha-Glucosidase Inhibitors

Alpha-glucosidase inhibitors are “diabetes pills” but not technically hypoglycemic agents because they do not have a direct effect on insulin secretion or sensitivity. These agents slow the digestion of starch in the small intestine, so that glucose from the starch of a meal enters the bloodstream more slowly, and can be matched more effectively by an impaired insulin response or sensitivity. These agents are effective by themselves only in the earliest stages of impaired glucose tolerance, but can be helpful in combination with other agents in type 2 diabetes.

Typical reductions in glycated hemoglobin (A1C) values are 0.5-1.0%.
miglitol
acarbose
voglibose

These medications have the potential to cause weight loss by lowering the amount of sugar metabolized.

### Peptide Analogs

### Injectable Incretin Mimetics

Incretins are insulin secretagogues. The two main candidate molecules that fulfill criteria for being an incretin are glucagon-like peptide-1 (GLP-1) and gastric inhibitory peptide (glucose-dependent insulinotropic peptide, GIP). Both GLP-1 and GIP are rapidly inactivated by the enzyme dipeptidyl peptidase-4 (DPP-4).

### Injectable Glucagon-Like Peptide Analogs and Agonists

Glucagon-like peptide (GLP) agonists bind to a membrane GLP receptor. As a consequence, insulin release from the pancreatic beta cells is increased. Endogenous GLP has a half-life of only a few minutes, thus an analogue of GLP would not be practical. As of 2019, the AACE lists GLP-1 agonists, along with SGLT2 inhibitors, as the most preferred anti-diabetic agents after metformin. Liraglutide in particular may be considered first-line in diabetic patients with cardiovascular disease, as it has received FDA approval for reduction of risk of major adverse cardiovascular events in patients with type 2 diabetes.
Exenatide (also Exendin-4, marketed as Byetta) is the first GLP-1 agonist approved for the treatment of type 2 diabetes. Exenatide is not an analogue of GLP but rather a GLP agonist. Exenatide has only 53% homology with GLP, which increases its resistance to degradation by DPP-4 and extends its half-life. Exenatide, together with liraglutide, led to greater weight loss than glucagon-like peptide analogues.
Liraglutide, a once-daily human analogue (97% homology), has been developed by Novo Nordisk under the brand name Victoza. The product was approved by the European Medicines Agency (EMEA) on Jul. 3, 2009, and by the U.S. Food and Drug Administration (FDA) on Jan. 25, 2010.
Taspoglutide is presently in Phase III clinical trials with Hoffman-La Roche.
Lixisenatide (Lyxumia) Sanofi Aventis
Semaglutide (Ozempic) (oral version is Rybelsus)
Dulaglutide (Trulicity)-once weekly
Albiglutide (Tanzeum)-once weekly
   These agents may also cause a decrease in gastric motility, responsible for the common side-effect of nausea, which tends to subside with time.

### Gastric Inhibitory Peptide Analogs

### Dipeptidyl Peptidase-4 Inhibitors

GLP-1 analogs resulted in weight loss and had more gastrointestinal side-effects, while in general dipeptidyl peptidase-4 (DPP-4) inhibitors were weight-neutral and increased risk for infection and headache, but both classes appear to present an alternative to other antidiabetic drugs. However, weight gain and/or hypoglycemia have been observed when dipeptidyl peptidase-4 inhibitors were used with sulfonylureas; effects on long-term health and morbidity rates are still unknown.

DPP-4 inhibitors increase blood concentration of the incretin GLP-1 by inhibiting its degradation by DPP-4. Examples are:
vildagliptin (Galvus) EU Approved 2008
sitagliptin (Januvia) FDA approved October 2006
saxagliptin (Onglyza) FDA Approved July 2009
linagliptin (Tradjenta) FDA Approved May 2, 2011
alogliptin
septagliptin
teneligliptin
gemigliptin (Zemiglo)
   DPP-4 inhibitors lowered hemoglobin A1C values by 0.74%, comparable to other antidiabetic drugs.

### Injectable Amylin Analogues

Amylin agonist analogues slow gastric emptying and suppress glucagon. They have all the incretins actions except stimulation of insulin secretion. As of 2007, pramlintide is the only clinically available amylin analogue. Like insulin, it is administered by subcutaneous injection. The most frequent and severe adverse effect of pramlintide is nausea, which occurs mostly at the beginning of treatment and gradually reduces. Typical reductions in A1C values are 0.5-1.0%.

### Glycosurics

SGLT-2 inhibitors block the re-uptake of glucose in the renal tubules, promoting loss of glucose in the urine. This causes both mild weight loss, and a mild reduction in blood sugar levels with little risk of hypoglycemia. Oral preparations may be available alone or in combination with other agents. Along with GLP-1 agonists, they are considered preferred second or third agents for type 2 diabetics sub-optimally controlled with metformin alone, according to most recent clinical practice guidelines. Because they are taken by mouth, rather than injected (like GLP-1 agonists), patients who are injection-averse may prefer these agents over the former. They may be considered first line in diabetic patients with cardiovascular disease, especially heart failure, as these medications have been shown to reduce the risk of hospitalization in patients with such comorbidities. Because they are not available as generic medications, however, cost may limit their feasibility for many patients. Furthermore, there has been growing evidence that the effectiveness and safety of this drug class could depend on genetic variability of the patients. Examples include:
Dapagliflozin
Canagliflozin
Empagliflozin
Remogliflozin
   The side effects of SGLT-2 inhibitors are derived directly from their mechanism of action; these include an increased risk of ketoacidosis, urinary tract infections, candidal vulvovaginitis, and hypoglycemia.

Many anti-diabetes drugs are available as generics. These include:
Sulfonylureas-glimepiride, glipizide, glyburide
Biguanides-metformin
Thiazolidinediones (Tzd)-pioglitazone, Actos generic
Alpha-glucosidase inhibitors-Acarbose
Meglitinides-nateglinide
Combination of sulfonylureas plus metformin-known by generic names of the two drugs
   No generics are available for dipeptidyl peptidase-4 inhibitors (Januvia, Onglyza), the glifozins, the incretins and various combinations.

### Exemplary Covid-19 Therapies

In certain aspects, the methods of the present disclosure further comprise recommending, prescribing, and/or administering to a subject (e.g., COVID-19 subject) determined to be at risk of developing diabetes, a COVID-19 therapy. In some embodiments, a COVID-19 therapy (e.g., antiviral therapy) comprises remdesivir, PF-07321332, molnupiravir (Lagevrio), MitoQ, a cell-derived therapeutic exosome, berzosertib, Favipiravir, lopinavir/ritonavir with or without IFN-beta-1a, ASC-09 and ritonavir, CD24Fc, Bamlanivimab and/oretesevimab, Bebtelovimab, Casirivimab/imdevimab (REGEN-COV, Ronapreve), Regdanvimab (Regkirona), Sotrovimab, Tixagevimab AZD8895) and/or cilgavimab (AZD1061) (collectively called Evusheld), Nirmatrelvir/ritonavir (Paxlovid), baricitinib, ensovibep, convalescent plasma, tocilizumab (Actemra), lenzilumab, Dapagliflozin, Apabetalone, Sarilumab, Sabizabulin, or any combination thereof.

### Pharmaceutical Composition

The compositions and methods of the present invention may be utilized to treat an individual in need thereof. In preferred embodiments, the composition or the compound of a therapy (e.g., COVID-19 therapy, diabetes therapy) is preferably administered as a pharmaceutical composition comprising, for example, a compound and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Exemplary Embodiments

1. A method of identifying a COVID-19-positive subject at risk for developing diabetes, the method comprising:
(a) determining the level of a miRNA in a sample from the COVID-19-positive subject; and
(b) comparing the level of the miRNA to a control, wherein a significantly higher level of the miRNA relative to the control indicates that the COVID-19-positive subject is at risk for developing diabetes, and wherein the sample comprises an endothelial cell extracellular vesicle (EV).
   2. The method of 1, wherein said significantly higher level of the miRNA comprises an at least 20% increase in the level of the miRNA
   3. The method of 1 or 2, wherein said significantly higher level of the miRNA comprises at least 50% increase in the level of the miRNA.
   4. The method of any one of 1-3, further comprising recommending, prescribing, and/or administering a therapy to prevent or treat diabetes to the COVID-19-positive subject determined to be at risk for developing diabetes.
   5. A method of preventing or treating diabetes in a COVID-19-positive subject, the method comprising
(a) identifying a COVID-19-positive subject at risk for developing diabetes according to the method of any one of 1-3; and
(b) administering to the subject a therapy to prevent or treat diabetes.
   6. The method of 4 or 5, wherein the therapy to prevent or treat diabetes comprises insulin, biguanides, thiazolidinediones, Lyn kinase activator, secretagogues, alpha-glucosidase inhibitor, peptide analog, glycosurics, or any combination thereof.
   7. The method of any one of 1-6, further comprising administering a COVID-19 therapy to the subject.
   8. The method of 7, wherein the COVID-19 therapy comprises remdesivir, PF-07321332, molnupiravir (Lagevrio), MitoQ, a cell-derived therapeutic exosome, berzosertib, Favipiravir, lopinavir/ritonavir with or without IFN-beta-1a, ASC-09 and ritonavir, CD24Fc, Bamlanivimab and/oretesevimab, Bebtelovimab, Casirivimab/imdevimab (REGEN-COV, Ronapreve), Regdanvimab (Regkirona), Sotrovimab, Tixagevimab AZD8895) and/or cilgavimab (AZD1061) (collectively called Evusheld), Nirmatrelvir/ritonavir (Paxlovid), baricitinib, ensovibep, convalescent plasma, tocilizumab (Actemra), lenzilumab, Dapagliflozin, Apabetalone, Sarilumab, Sabizabulin, or any combination thereof.
   9. The method of any one of 1-8, wherein the control comprises the miRNA level in
(a) a sample from a healthy subject, a COVID-19-negative subject, a subject without diabetes, or a COVID-19-positive subject without diabetes; or
(b) a portion or all of pooled samples from one or more subjects of (a).
   10. The method of any one of 1-8, wherein the control is a pre-determined level of the miRNA.
   11. The method of any one of 1-10, wherein the sample comprises body fluid of the subject, optionally plasma of the subject.
   12. The method of any one of 1-11, wherein the sample comprises a CD31-positive extracellular vesicle.
   13. The method of any one of 1-12, further comprising fractionating EVs from the sample before detecting the level of the miRNA.
   14. The method of any one of 1-13, further comprising isolating the endothelial cell extracellular vesicle (EC-EVs) and/or a CD31-positive extracellular vesicle from the sample before detecting the level of the miRNA.
   15. The method of any one of 1-14, further comprising reverse transcribing the miRNA into a cDNA before detecting the level of the miRNA.
   16. The method of any one of 1-15, wherein the level of the miRNA is detected by a method comprising: multiplex bead-based assays, RNA-seq, next generation sequencing, sequencing, mass spectrometry (e.g., RNA sequencing by LC-MS, cDNA sequencing by LC-MS), microarray, Southern blotting of the cDNA of miRNA, Northern blotting, PCR, RT-PCR, real-time PCR (e.g., TaqMan®), any variation thereof, or any combination of two or more thereof.
   17. The method of any of claims 1-16, wherein the miRNA is miR-34a.
   18. The method of any one of 1-17, wherein the COVID-19 subject has long COVID.
   19. The method of any one of 1-18, wherein the subject is a mammal.
   20. The method of any one of 1-19, wherein the subject is a dog, a cat, or a human, optionally a human.

### EXAMPLES

### Example 1: Isolation of CD31+ EVs

Subject blood samples are obtained and centrifuged at 753 g at 4° C. to obtain platelet-poor EDTA plasma and stored at −80° C. within 3 h from blood collection.

Plasma EDTA samples are allowed to thaw at room temperature. Appropriate volume (100 μL when used for singular miRNA dosage) is diluted with an equal amount of PBS. For removal of apoptotic bodies and residual cellularity, sample are precleared by two subsequent centrifugations at 4° C.: one at 2,000 g for 30 min and the following at 10,000 g for 45 min. Supernatant is diluted with PBS to reach 500 μL volume and then mixed with FcR Blocking Reagent (20 μL) provided in the commercially available kit for endothelial cells isolation (130-091-935; Miltenyi Biotec). After vortexing, 20 μL CD31 MicroBeads (130-091-935; Miltenyi Biotec) are added to the suspension and incubated at 4° C. in the dark for 30 min. Appropriate (according to reaction volume) columns are mounted on the magnetic field and activated with PBS. After incubation, the mixture is loaded onto the column to allow separation. After three washes with 500 μL PBS, the column is removed from the magnetic support and CD31+ EVs are eluted in 500 μL PBS with the help of a plunger. As a negative control, isotype control beads (DynaBeads M-280) and no beads (equal amount of PBS) are used for parallel isolation of EVs for testing for eventual nonspecific bindings of EVs and subjected to MACSPlex comparison.

### Example 2: Isolation of EVs Through Ultracentrifugation

An aliquot of 1 mL plasma is precleared as indicated above and then the supernatant is diluted with PBS and subjected to ultracentrifugation (UC) at 120,000 g (4° C.) in a Thermo Scientific S110AT rotor in a Sorvall MX 150 ultracentrifuge for 1.5 h. Pellets are resuspended in PBS and ultracentrifuged again at 120,000 g for an additional 1.5 h. The final pellets are resuspended in 500 μL PBS.

### Example 3: Cytofluorimetric Detection of EV Markers

A commercially available (cat. no. 130-108-813, MACSPlex Exosome Kit; Miltenyi Biotec) kit is used for cytofluorimetric detection of a large range of markers in isolated EVs. Briefly, EVs isolated starting from the same amount of plasma are prepared as described in the manufacturer protocol. The multiplex bead-based platform is analyzed by flow cytometry with use of a BD FACSCanto II flow cytometer with the corresponding software (Becton, Dickinson and Company, Franklin Lakes, NJ) equipped with a 488-nm and a 640-nm laser. Fluorescence emission is collected by 530/30 nm, 585/42 nm, and 660/20 nm bandpass filters. At least 1,000 beads per sample are examined, and mean fluorescence intensity is determined with use of BD FACSDiva 6.1 software. Background signals are determined by analysis of beads incubated only with the respective staining antibodies and subtracted from the signals obtained for beads incubated with EVs and stained with the corresponding antibody. The multiplex bead-based platform includes setup beads for flow cytometer setup.

### Example 4: RNA Extraction and miRNA Profiling

Plasma samples from the subjects are pooled to reach 1 mL. CD31+ EVs are isolated from control preparations and COVID-19 patient preparations. RNA is extracted with a commercial kit known to enrich small RNA species (Norgen Biotek Corporation). The same amount of RNA is converted to cDNA by priming with a mixture of looped primers according to the manufacturer's instructions (Megaplex kit; Applied Biosystems). cDNA (9 μL) is used for mature miRNA profiling by a real-time PCR instrument equipped with a 384-well reaction plate and human miRNA array pool A containing 367 different human miRNA assays in addition to selected small nucleolar RNAs and negative controls (non-human miRNAs). Only miRNAs expressed in more than one sample are included in the analysis. 2-Ct of the average values of each miRNA are used to build the heat map comparing control and COVID-19 patients with the ClustVis web tool (World Wide Web at biit.cs.ut.ee/clustvis/).

### Example 5: Single miRNA Quantitation

For single miRNA quantification, CD31+ EVs are isolated from 100 μL plasma. After mixing with lysis buffer and before loading to the RNA separation column (Norgen Biotek Corporation), the synthetic non-human miRNA is spiked into plasma before RNA extraction. Only samples with the synthetic non-human miRNA recovery >95% are used in subsequent analyses. Reverse transcription and miR-34a amplification are performed as previously described and as known in the art. Relative expression correspond to the 2^{-ΔCt} value. The miRNA expression levels are normalized by the spiked synthetic non-human miRNA.

### Example 6: Clinical Observations

We previously identified a link between COVID-19 and endothelial dysfunction (Refs 12, 13, and 53), and our view was later confirmed by other investigators, associating the systemic manifestations of the disease to a direct or indirect involvement of the endothelium (Refs 14-16 and 54-66). Indeed, endothelial cells (ECs) express all co-factors necessary for the internalization of SARS-COV-2 in host cells, including angiotensin converting enzyme 2 (ACE2), transmembrane protease serine 2, cathepsins B and D, neuropilin-1, vimentin, and others, thereby representing a natural target of SARS-COV-2 (as described in Refs 80-83).

Furthermore, the systemic inflammatory viral reaction observed in patients affected by COVID-19 has been shown to be linked to endothelial dysfunction (Refs 17 and 67-69). COVID-19 affects not only the epithelial cells of the lung parenchyma, but also ECs across the whole body, thus leading to a generalized endothelial damage. Such a damage, caused directly by SARS-COV-2 infection and/or by the ensuing cytokine storm, can shift the vascular equilibrium towards an altered vascular tone, and an increased permeabilization; most of these findings have been substantiated from autopsies of COVID-19 patients since the outbreak of the pandemic (Ref 15). Further supporting our theory of a central role of ECs in COVID-19, clinical trials testing whether interventions that ameliorate endothelial dysfunction can have beneficial effects in COVID-19 patients are ongoing and preliminary interim results are very encouraging (Refs 18 and 70-71).

In a preliminary assay comparing circulating levels of endothelial cell extracellular vesicles (EC-EVs) of COVID-19 patients who developed diabetes to COVID-19 patients who did not develop diabetes, we identified miR-34a as one of the top upregulated miRNAs. Therefore, we hypothesized an association between plasma levels of EC-EV miR-34a and the onset of diabetes in patients hospitalized for COVID-19.

We obtained plasma from 388 patients hospitalized for COVID-19, consecutively enrolled from January 2021 to January 2022 at the “Ospedali dei Colli”. The diagnosis of diabetes was defined according to the guidelines of the American Diabetes Association (Ref 20). The study was performed in accordance with the ethical standards laid down in the 1964 Declaration of Helsinki and its later amendments.

Inclusion criteria: age >18 years old; willingness to participate (informed consent signed by each patient or an authorized representative); diagnosis of COVID-19 confirmed by RT-qPCR on nasal swab, as described in (Refs 18, 19, 71, and 72).

Exclusion criteria: patients for whom admission blood samples were unavailable, patients with cancer, patients with a pre-existent diagnosis of diabetes or prediabetes (Ref 73), patients who were not treated with steroids, and patients who received the first diagnosis of diabetes less than 30 days after discontinuation of steroid-based treatments were excluded.

We excluded 73 patients with a preexistent diagnosis of diabetes, patients with cancer, patients who were not treated with steroids, or unavailability of admission blood samples; thus, the study was conducted in 315 subjects, with a median follow-up of 12 months. A SARS-COV-2 test (RT-qPCR) was performed in all subjects.

EC-EVs were extracted from the plasma collected from these patients via serial centrifugation and CD31+ magnetic isolation (as described and validated in Refs 74, 75, 19) and DNA was extracted as reported in (Refs 19, 76, and 77); EC-EV miRNA-34a levels were quantified via droplet digital polymerase chain reaction (ddPCR) using a QX200 ddPCR System (BIO-RAD, Hercules, CA) following the manufacturer's instructions, as we described in (Ref 19). A SARS-COV-2 test (RT-qPCR) was performed in all subjects to confirm or rule out the COVID-19 diagnosis. Blood levels of Interleukin-6 (IL-6), D-dimer, high-sensitivity C Reactive Protein (hs-CRP), and Tumor Necrosis Factor α (TNFα) were measured in all patients on admission as described in (Refs 18 and 19).

All data were analyzed using the SPSS software (version 29.0; SPSS, IBM, Armonk, NY, USA), establishing a significant difference at a p-value <0.05. Data are expressed as means±SD or numbers and percentages. The unpaired 2-tailed t-test using (when appropriate) Welch's correction for unequal variances was performed. The chi-squared test was applied to compare categorical variables. A multivariable linear regression analysis was used to assess the association between miR-34a and new-onset diabetes, adjusting for potential confounders. Receiver operating characteristic (ROC) curves were analyzed to identify the optimal cut-off value of miR-34a levels, calculating the Youden's index to integrate sensitivity and specificity information as described in (Refs 102 and 79).

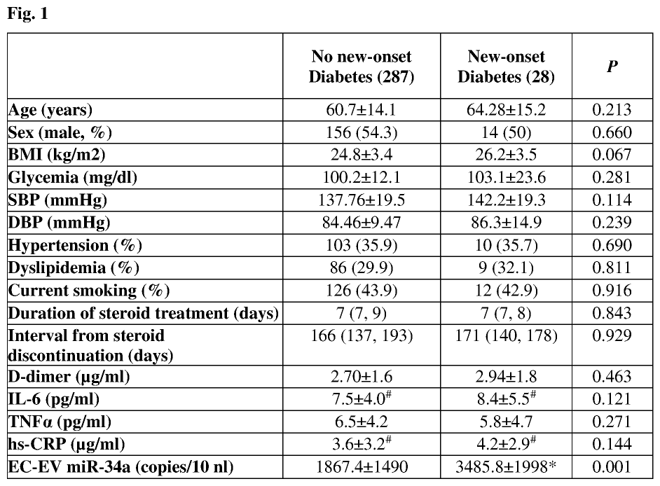

Clinical parameters of our population are reported in FIG. 1. New onset diabetes was diagnosed in 28 COVID-19 patients. No significant differences in co-morbidities and in therapeutic management were observed. As per our exclusion criteria, all subjects received steroids as standard therapy for hospitalized COVID-19 patients.

We found that circulating levels of EC-EV the miR-34a were significantly upregulated (P<0.001) in patients with vs without new-onset diabetes among COVID-19 patients, but not when examining subjects without COVID-19 (FIG. 1). Strikingly, using a stepwise multiple regression analysis, adjusting for age, hypertension, dyslipidemia, diabetes, and D-dimer, the association between EC-EV the miRNA and new-onset diabetes in COVID-19 patients was confirmed (P<0.001).

Applying ROC curves, we determined that 3300 copies/10 nl was the optimal cut-off value for miR-34a levels in order to predict new-onset diabetes, yielding the following results: sensitivity 71.43% (95% C.I.: 51.33% to 86.78%), specificity 98.61% (95% C.I.: 96.47% to 99.62%), positive predictive value 83.33% (95% C.I.: 64.76% to 93.15%), and negative predictive value 97.25% (95% C.I.: 95.17% to 98.45%).

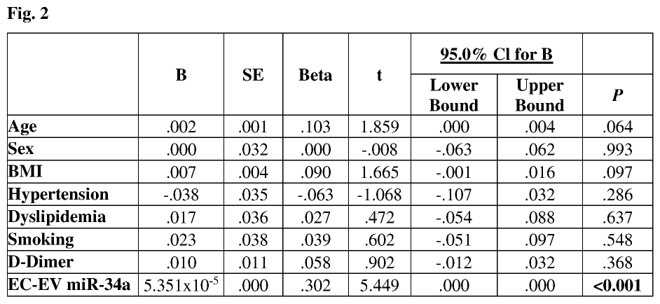

Using a stepwise multiple regression analysis, adjusting for age, sex, BMI, hypertension, dyslipidemia, smoking status, and D-dimer (FIG. 2), the association between EC-EV miR-34a and new-onset diabetes in COVID-19 patients was confirmed (P<0.001).

This is the first study showing an association between EC-EV noncoding RNA and new-onset diabetes in COVID-19 patients.

In conclusion, we identified a significant association linking EC-EV the miRNA and new onset diabetes. miR-34a was identified as a novel and independent biomarker of disease and biomarker-centric approaches have been shown to be crucial in drug development (Ref 103).

### REFERENCES

1. Zhu L, She Z G, Cheng X, Qin J J, Zhang X J, Cai J, Lei F, Wang H, Xie J, Wang W, Li H, Zhang P, Song X, Chen X, Xiang M, Zhang C, Bai L, Xiang D, Chen M M, Liu Y, Yan Y, Liu M, Mao W, Zou J, Liu L, Chen G, Luo P, Xiao B, Zhang C, Zhang Z, Lu Z, Wang J, Lu H, Xia X, Wang D, Liao X, Peng G, Ye P, Yang J, Yuan Y, Huang X, Guo J, Zhang B H and Li H. Association of Blood Glucose Control and Outcomes in Patients with COVID-19 and Preexisting Type 2 Diabetes. Cell Metab. 2020; 31:1068-1077 e3.
2. Accili D. Can COVID-19 cause diabetes? Nat Metab. 2021; 3:123-125.
3. Rubino F, Amiel S A, Zimmet P, Alberti G, Bornstein S, Eckel R H, Mingrone G, Boehm B, Cooper M E, Chai Z, Del Prato S, Ji L, Hopkins D, Herman W H, Khunti K, Mbanya J C and Renard E. New-Onset Diabetes in Covid-19. N Engl J Med. 2020; 383:789-790.
4. Barrett C E, Koyama A K, Alvarez P, Chow W, Lundeen E A, Perrine C G, Pavkov M E, Rolka D B, Wiltz J L, Bull-Otterson L, Gray S, Boehmer T K, Gundlapalli A V, Siegel D A, Kompaniyets L, Goodman A B, Mahon B E, Tauxe R V, Remley K and Saydah S. Risk for Newly Diagnosed Diabetes >30 Days After SARS-COV-2 Infection Among Persons Aged <18 Years-United States, Mar. 1, 2020-Jun. 28, 2021. MMWR Morb Mortal Wkly Rep. 2022; 71:59-65.
5. Kamrath C, Rosenbauer J, Eckert A J, Pappa A, Reschke F, Rohrer T R, Monkemoller K, Wurm M, Hake K, Raile K and Holl R W. Incidence of COVID-19 and Risk of Diabetic Ketoacidosis in New-Onset Type 1 Diabetes. Pediatrics. 2021.
6. Sathish T, Anton M C and Sivakumar T. New-onset diabetes in “long COVID”. J Diabetes. 2021; 13:693-694.
7. Raveendran A V and Misra A. Post COVID-19 Syndrome (“Long COVID”) and Diabetes: Challenges in Diagnosis and Management. Diabetes Metab Syndr. 2021; 15:102235.
8. Feldman E L, Savelieff M G, Hayek S S, Pennathur S, Kretzler M and Pop-Busui R. COVID-19 and Diabetes: A Collision and Collusion of Two Diseases. Diabetes. 2020; 69:2549-2565.
9. Montefusco L, Ben Nasr M, D'Addio F, Loretelli C, Rossi A, Pastore I, Daniele G, Abdelsalam A, Maestroni A, Dell'Acqua M, Ippolito E, Assi E, Usuelli V, Seelam A J, Fiorina R M, Chebat E, Morpurgo P, Lunati M E, Bolla A M, Finzi G, Abdi R, Bonventre J V, Rusconi S, Riva A,Corradi D, Santus P, Nebuloni M, Folli F, Zuccotti G V, Galli M and Fiorina P. Acute and long-term disruption of glycometabolic control after SARS-COV-2 infection. Nat Metab. 2021.
10. Al-Aly Z, Xie Y and Bowe B. High-dimensional characterization of post-acute sequelae of COVID-19. Nature. 2021; 594:259-264.
11. Ayoubkhani D, Khunti K, Nafilyan V, Maddox T, Humberstone B, Diamond I and Banerjee A. Post-covid syndrome in individuals admitted to hospital with covid-19: retrospective cohort study. BMJ. 2021; 372:n693.
12. Sardu C, Gambardella J, Morelli M B, Wang X, Marfella R and Santulli G. Hypertension, Thrombosis, Kidney Failure, and Diabetes: Is COVID-19 an Endothelial Disease? A Comprehensive Evaluation of Clinical and Basic Evidence. J Clin Med. 2020; 9.
13. Santulli G, Morelli M and Gambardella J. Is Endothelial Dysfunction the Concealed Cornerstone of COVID-19? BMJ. 2020; 368.
14. Kelliher S, Weiss L, Cullivan S, O'Rourke E, Murphy C A, Toolan S, Lennon A, Szklanna P B, corner S P, Macleod H, Le Chevillier A, Gaine S, O'Reilly K M A, McCullagh B, Stack J, Maguire P B, Ni Ainle F and Kevane B. Non-severe COVID-19 is associated with endothelial damage and hypercoagulability despite pharmacological thromboprophylaxis. J Thromb Haemost. 2022.
15. Otifi H M and Adiga B K. Endothelial Dysfunction in Covid-19. Am J Med Sci. 2022.
16. Qin Z, Liu F, Blair R, Wang C, Yang H, Mudd J, Currey J M, Iwanaga N, He J, Mi R, Han K, Midkiff C C, Alam M A, Aktas B H, Heide R S V, Veazey R, Piedimonte G, Maness N J, Ergun S, Mauvais-Jarvis F, Rappaport J, Kolls J K and Qin X. Endothelial cell infection and dysfunction, immune activation in severe COVID-19. Theranostics. 2021; 11:8076-8091.
17. Gustafson D, Raju S, Wu R, Ching C, Veitch S, Rathnakumar K, Boudreau E, Howe K L and Fish J E. Overcoming Barriers: The Endothelium as a Linchpin of Coronavirus Disease 2019 Pathogenesis? Arterioscler Thromb Vasc Biol. 2020; 40:1818-1829.
18. Fiorentino G, Coppola A, Izzo R, Annunziata A, Bernardo M, Lombardi A, Trimarco V, Santulli G and Trimarco B. Effects of adding L-arginine orally to standard therapy in patients with COVID-19: A randomized, double-blind, placebo-controlled, parallel-group trial. Results of the first interim analysis. EclinicalMedicine. 2021; 40:101125.
19. Gambardella J, Coppola A, Izzo R, Fiorentino G, Trimarco B and Santulli G. Role of endothelial miR-24 in COVID-19 cerebrovascular events. Crit Care. 2021; 25:306.
20. Draznin B, Aroda V R, Bakris G, Benson G, Brown F M, Freeman R, Green J, Huang E, Isaacs D, Kahan S, Leon J, Lyons S K, Peters A L, Prahalad P, Reusch JEB, Young-Hyman D, Das S and Kosiborod M. Classification and Diagnosis of Diabetes: Standards of Medical Care in Diabetes-2022. Diabetes Care. 202245:S17-S38.
21. Kastora S, Patel M, Carter B, Delibegovic M and Myint P K (2022) Impact of diabetes on COVID-19 mortality and hospital outcomes from a global perspective: An umbrella systematic review and meta-analysis. Endocrinol Diabetes Metab:e338.
22. Kazakou P, Lambadiari V, Ikonomidis I, Kountouri A, Panagopoulos G, Athanasopoulos S, Korompoki E, Kalomenidis I, Dimopoulos M A and Mitrakou A (2022) Diabetes and COVID-19; A Bidirectional Interplay. Front Endocrinol (Lausanne) 13:780663.
23. Kim D, Alshuwaykh O, Sandhu K K, Dennis B B, Cholankeril G and Ahmed A (2022) Trends in All-Cause and Cause-Specific Mortality Among Individuals With Diabetes Before and During the COVID-19 Pandemic in the U.S. Diabetes Care.
24. Sathish T, Kapoor N, Cao Y, Tapp R J and Zimmet P (2021b) Proportion of newly diagnosed diabetes in COVID-19 patients: A systematic review and meta-analysis. Diabetes Obes Metab 23:870-874.
25. Khunti K, Del Prato S, Mathieu C, Kahn S E, Gabbay R A and Buse J B (2021) COVID-19, Hyperglycemia, and New-Onset Diabetes. Diabetes Care 44:2645-2655.
26. Metwally A A, Mehta P, Johnson B S, Nagarjuna A and Snyder M P (2021) COVID-19-Induced New-Onset Diabetes: Trends and Technologies. Diabetes 70:2733-2744.
27. Nassar M, Nso N, Baraka B, Alfishawy M, Mohamed M, Nyabera A and Sachmechi I (2021) The association between COVID-19 and type 1 diabetes mellitus: A systematic review. Diabetes Metab Syndr 15:447-454.
28. Lai H, Yang M, Sun M, Pan B, Wang Q, Wang J, Tian J, Ding G, Yang K, Song X and Ge L (2022) Risk of incident diabetes after COVID-19 infection: A systematic review and meta-analysis. Metabolism 137:155330.
29. Rahmati M, Keshvari M, Mirnasuri S, Yon D K, Lee S W, Il Shin J and Smith L (2022) The global impact of COVID-19 pandemic on the incidence of pediatric new-onset type 1 diabetes and ketoacidosis: A systematic review and meta-analysis. J Med Virol 94:5112-5127.
30. Rathmann W, Kuss O and Kostev K (2022) Incidence of newly diagnosed diabetes after Covid-19. Diabetologia 65:949-954.
31. Ssentongo P, Zhang Y, Witmer L, Chinchilli V M and Ba D M (2022) Association of COVID-19 with diabetes: a systematic review and meta-analysis. Sci Rep 12:20191.
32. Steenblock C, Hassanein M, Khan E G, Yaman M, Kamel M, Barbir M, Lorke D E, Rock J A, Everett D, Bejtullah S, Heimerer A, Tahirukaj E, Beqiri P and Bornstein S R (2022) Diabetes and COVID-19: Short- and Long-Term Consequences. Horm Metab Res 54:503-509.
33. Wander P L, Lowy E, Beste L A, Tulloch-Palomino L, Korpak A, Peterson A C, Kahn S E and Boyko E J (2022) The Incidence of Diabetes Among 2,777,768 Veterans With and Without Recent SARS-COV-2 Infection. Diabetes Care 45:782-788.
34. Xie Y and Al-Aly Z (2022) Risks and burdens of incident diabetes in long COVID: a cohort study. Lancet Diabetes Endocrinol 10:311-321.
35. Zhang T, Mei Q, Zhang Z, Walline J H, Liu Y, Zhu H and Zhang S (2022) Risk for newly diagnosed diabetes after COVID-19: a systematic review and meta-analysis. BMC Med 20:444.
36. Abumayyaleh M, Nunez Gil I J, Viana L M C, Raposeiras Roubin S, Romero R, Alfonso-Rodriguez E, Uribarri A, Feltes G, Becerra-Munoz V M, Santoro F, Pepe M, Castro Mejia A F, Signes-Costa J, Gonzalez A, Marin F, Lopez-Pais J, Manzone E, Vazquez Cancela O, Paeres C E, Masjuan A L, Velicki L, Weiss C, Chipayo D, Fernandez-Ortiz A, El-Battrawy I, Akin I and investigators HC-(2023) Post-COVID-19 syndrome and diabetes mellitus: a propensity-matched analysis of the International HOPE-II COVID-19 Registry. Front Endocrinol (Lausanne) 14:1167087.
37. Ali H, Abu-Farha M and Hofmann S (2023) Editorial: COVID-19 and diabetes, volume II. Front Endocrinol (Lausanne) 14:1136997.
38. Bellia C, Andreadi A, D'Ippolito I, Scola L, Barraco S, Meloni M, Lauro D and Bellia A (2023) Prevalence and risk of new-onset diabetes mellitus after COVID-19: a systematic review and meta-analysis. Front Endocrinol (Lausanne) 14:1215879.
39. Cefalu W T (2023) COVID-19 and Rising Incidence of Diabetes: Despite Evolving Data, an Enigma Still to Be Solved. Diabetes Care 46:913-915.
40. Chourasia P, Goyal L, Kansal D, Roy S, Singh R, Mahata I, Sheikh A B and Shekhar R (2023) Risk of New-Onset Diabetes Mellitus as a Post-COVID-19 Condition and Possible Mechanisms: A Scoping Review. J Clin Med 12.
41. Gorchane A, Ach T, Sahli J, Abdelkrim A B, Mallouli M, Bellazreg F, Hachfi W, Chaieb M C and Ach K (2023) Uncovering the alarming rise of diabetic ketoacidosis during COVID-19 pandemic: a pioneer African study and review of literature. Front Endocrinol (Lausanne) 14:1234256.
42. Izzo R, Pacella D, Trimarco V, Manzi M V, Lombardi A, Piccinocchi R, Gallo P, Esposito G, Lembo M, Piccinocchi G, Morisco C, Santulli G and Trimarco B (2023) Incidence of type 2 diabetes before and during the COVID-19 pandemic in Naples, Italy: a longitudinal cohort study. EclinicalMedicine 66:102345.
43. Kim S H, Arora I, Hsia D S, Knowler W C, LeBlanc E, Mylonakis E, Pratley R and Pittas A G (2023) New-Onset Diabetes After COVID-19. J Clin Endocrinol Metab 108:e1164-e1174.
44. Li Y, Peng L and Gu W (2023) The published trend of studies on COVID-19 and diabetes: bibliometric analysis. Front Endocrinol (Lausanne) 14:1248676.
45. Naveed Z, Velasquez Garcia H A, Wong S, Wilton J, McKee G, Mahmood B, Binka M, Rasali D and Janjua N Z (2023) Association of COVID-19 Infection With Incident Diabetes. JAMA Netw Open 6:e238866.
46. O'Mahoney L L, Routen A, Gillies C, Ekezie W, Welford A, Zhang A, Karamchandani U, Simms-Williams N, Cassambai S, Ardavani A, Wilkinson T J, Hawthorne G, Curtis F, Kingsnorth A P, Almaqhawi A, Ward T, Ayoubkhani D, Banerjee A, Calvert M, Shafran R, Stephenson T, Sterne J, Ward H, Evans R A, Zaccardi F, Wright S and Khunti K (2023) The prevalence and long-term health effects of Long Covid among 55ospitalized and non-hospitalised populations: A systematic review and meta-analysis. EclinicalMedicine 55:101762.
47. Weiss A, Donnachie E, Beyerlein A, Ziegler A G and Bonifacio E (2023) Type 1 Diabetes Incidence and Risk in Children With a Diagnosis of COVID-19. JAMA 329:2089-2091.
48. Zhou L, Qu H, Zhang Q, Hu J and Shou L (2023) Case report: Fulminant type 1 diabetes following paucisymptomatic SARS-COV-2 infection during late pregnancy. Front Endocrinol (Lausanne) 14:1168927.
49. Pantea Stoian A, Bica I C, Salmen T, Al Mahmeed W, Al-Rasadi K, Al-Alawi K, Banach M, Banerjee Y, Ceriello A, Cesur M, Cosentino F, Firenze A, Galia M, Goh S Y, Janez A, Kalra S, Kapoor N, Kempler P, Lessan N, Lotufo P, Mikhailidis D P, Nibali L, Papanas N, Powell-Wiley T, Rizvi A A, Sahebkar A, Santos R D, Toth P P, Viswanathan V, Rizzo M and Cardiometabolic Panel of International Experts on Syndemic C (2024) New-Onset Diabetes Mellitus in COVID-19: A Scoping Review. Diabetes Ther 15:33-60.
50. Iqbal F M, Lam K, Sounderajah V, Clarke J M, Ashrafian H and Darzi A (2021) Characteristics and predictors of acute and chronic post-COVID syndrome: A systematic review and meta-analysis. EclinicalMedicine 36:100899.
51. Narayan KMV and Staimez L R (2022) Rising diabetes diagnosis in long COVID. Lancet Diabetes Endocrinol.
52. Scherer P E, Kirwan J P and Rosen C J (2022) Post-acute sequelae of COVID-19: A metabolic perspective. Elife 11.
53. Gambardella J, Khondkar W, Morelli M B, Wang X, Santulli G and Trimarco V (2020) Arginine and Endothelial Function. Biomedicines 8:277.
54. D'Agnillo F, Walters K A, Xiao Y, Sheng Z M, Scherler K, Park J, Gygli S, Rosas L A, Sadtler K, Kalish H, Blatti C A, 3^{rd}, Zhu R, Gatzke L, Bushell C, Memoli M J, O'Day S J, Fischer T D, Hammond T C, Lee R C, Cash J C, Powers M E, O'Keefe G E, Butnor K J, Rapkiewicz A V, Travis W D, Layne S P, Kash J C and Taubenberger J K (2021) Lung epithelial and endothelial damage, loss of tissue repair, inhibition of fibrinolysis, and cellular senescence in fatal COVID-19. Sci Transl Med 13:eabj7790.
55. Gelzo M, Cacciapuoti S, Pinchera B, De Rosa A, Cernera G, Scialo F, Comegna M, Mormile M, Fabbrocini G, Parrella R, Corso G, Gentile I and Castaldo G (2021) Further Findings Concerning Endothelial Damage in COVID-19 Patients. Biomolecules 11.
56. Higashikuni Y, Liu W, Obana T and Sata M (2021) Pathogenic Basis of Thromboinflammation and Endothelial Injury in COVID-19: Current Findings and Therapeutic Implications. Int J Mol Sci 22.
57. Kalicinska E, Biernat M, Rybka J, Zinczuk A, Janocha-Litwin J, Rosiek-Biegus M, Morawska M, Waszczuk-Gajda A, Drozd-Sokolowska J, Szukalski L, Rymko M, Jablonowska P, Simon K and Wrobel T (2021) Endothelial Activation and Stress Index (EASIX) as an Early Predictor for Mortality and Overall Survival in Hematological and Non-Hematological Patients with COVID-19: Multicenter Cohort Study. J Clin Med 10.
58. Beltran-Camacho L, Eslava-Alcon S, Rojas-Torres M, Sanchez-Morillo D, Martinez-Nicolas M P, Martin-Bermejo V, de la Torre I G, Berrocoso E, Moreno J A, Moreno-Luna R and Duran-Ruiz M C (2022) The serum of COVID-19 asymptomatic patients up-regulates proteins related to endothelial dysfunction and viral response in circulating angiogenic cells ex-vivo. Mol Med 28:40.
59. Casciola-Rosen L, Thiemann D R, Andrade F, Trejo-Zambrano M I, Leonard E K, Spangler J B, Skinner N E, Bailey J, Yegnasubramanian S, Wang R, Vaghasia A M, Gupta A, Cox A L, Ray S C, Linville R M, Guo Z, Searson P C, Machamer C E, Desiderio S, Sauer L M, Laeyendecker O, Garibaldi B T, Gao L, Damarla M, Hassoun P M, Hooper J E, Mecoli C A, Christopher-Stine L, Gutierrez-Alamillo L, Yang Q, Hines D, Clarke W A, Rothman R E, Pekosz A, Fenstermacher K Z, Wang Z, Zeger S L and Rosen A (2022) IgM anti-ACE2 autoantibodies in severe COVID-19 activate complement and perturb vascular endothelial function. JCI Insight.
60. Haffke M, Freitag H, Rudolf G, Seifert M, Doehner W, Scherbakov N, Hanitsch L, Wittke K, Bauer S, Konietschke F, Paul F, Bellmann-Strobl J, Kedor C, Scheibenbogen C and Sotzny F (2022) Endothelial dysfunction and altered endothelial biomarkers in patients with post-COVID-19 syndrome and chronic fatigue syndrome (ME/CFS). J Transl Med 20:138.
61. Ma Z, Yang K Y, Huang Y and Lui K O (2022) Endothelial contribution to COVID-19: an update on mechanisms and therapeutic implications. J Mol Cell Cardiol 164:69-82.
62. Maldonado F, Morales D, Diaz-Papapietro C, Valdes C, Fernandez C, Valls N, Lazo M, Espinoza C, Gonzalez R, Gutierrez R, Jara A, Romero C, Cerda O and Caceres M (2022) Relationship Between Endothelial and Angiogenesis Biomarkers Envisage Mortality in a Prospective Cohort of COVID-19 Patients Requiring Respiratory Support. Front Med (Lausanne) 9:826218.
63. Montiel V, Lobysheva I, Gerard L, Vermeersch M, Perez-Morga D, Castelein T, Mesland J B, Hantson P, Collienne C, Gruson D, van Dievoet M A, Persu A, Beauloye C, Dechamps M, Belkhir L, Robert A, Derive M, Laterre P F, Danser AHJ, Wittebole X and Balligand J L (2022) Oxidative stress-induced endothelial dysfunction and decreased vascular nitric oxide in COVID-19 patients. EbioMedicine 77:103893.
64. Santoro L, Falsetti L, Zaccone V, Nesci A, Tosato M, Giupponi B, Savastano M C, Moroncini G, Gasbarrini A, Landi F, Santoliquido A and On Behalf Of Gemelli Against Covid-Post-Acute Care Study G (2022) Impaired Endothelial Function in Convalescent Phase of COVID-19: A 3 Month Follow Up Observational Prospective Study. J Clin Med 11.
65. Seitz A and Ong P (2022) Endothelial dysfunction in COVID-19: A potential predictor of long-COVID? Int J Cardiol 349:155-156.
66. Tarnawski A S and Ahluwalia A (2022) Endothelial cells and blood vessels are major targets for COVID-19-induced tissue injury and spreading to various organs. World J Gastroenterol 28:275-289.
67. Bernard I, Limonta D, Mahal L K and Hobman T C (2020) Endothelium Infection and Dysregulation by SARS-COV-2: Evidence and Caveats in COVID-19. Viruses 13.
68. Loo J, Spittle D A and Newnham M (2021) COVID-19, immunothrombosis and venous thromboembolism: biological mechanisms. Thorax 76:412-420.
69. Sabioni L, De Lorenzo A, Lamas C, Muccillo F, Castro-Faria-Neto H C, Estato V and Tibirica E (2021) Systemic microvascular endothelial dysfunction and disease severity in COVID-19 patients: Evaluation by laser Doppler perfusion monitoring and cytokine/chemokine analysis. Microvasc Res 134:104119.
70. Adebayo A, Varzideh F, Wilson S, Gambardella J, Eacobacci M, Jankauskas S S, Donkor K, Kansakar U, Trimarco V, Mone P, Lombardi A and Santulli G (2021) 1-Arginine and COVID-19: An Update. Nutrients 13.
71. Trimarco V, Izzo R, Lombardi A, Coppola A, Fiorentino G and Santulli G (2023) Beneficial effects of L-Arginine in patients hospitalized for COVID-19: New insights from a randomized clinical trial. Pharmacol Res 191:106702.
72. Trimarco V, Izzo R, Zanforlin A, Tursi F, Scarpelli F, Santus P, Pennisi A, Pelaia G, Mussi C, Mininni S, Messina N, Marazzi G, Maniscalco M, Mallardo M, Fazio G, Diana A, Capra Marzani M, Aloe T, Mone P, Trimarco B and Santulli G (2022) Endothelial dysfunction in long-COVID: New insights from the nationwide multicenter LINCOLN Study. Pharmacol Res 185:106486.
73. Mone P, De Gennaro S, Moriello D, Frullone S, D'Amelio R, Ferrante MNV, Marro A and Santulli G (2023a) Insulin resistance drives cognitive impairment in hypertensive pre-diabetic frail elders: the CENTENNIAL study. Eur J Prev Cardiol 30:1283-1288.
74. Morelli M B, Shu J, Sardu C, Matarese A and Santulli G (2019) Cardiosomal microRNAs Are Essential in Post-Infarction Myofibroblast Phenoconversion. Int J Mol Sci 21.
75. Wang X, Morelli M B, Matarese A, Sardu C and Santulli G (2020) Cardiomyocyte-derived exosomal microRNA-92a mediates post-ischemic myofibroblast activation both in vitro and ex vivo. ESC Heart Fail 7:284-288.
76. Jankauskas S S, Mone P, Avvisato R, Varzideh F, De Gennaro S, Salemme L, Macina G, Kansakar U, Cioppa A, Frullone S, Gambardella J, Di Mauro M, Tesorio T and Santulli G (2023) miR-181c targets Parkin and SMAD7 in human cardiac fibroblasts: Validation of differential microRNA expression in patients with diabetes and heart failure with preserved ejection fraction. Mech Ageing Dev 212:111818.
77. Mone P, Lombardi A, Kansakar U, Varzideh F, Jankauskas S S, Pansini A, Marzocco S, De Gennaro S, Famiglietti M, Macina G, Frullone S and Santulli G (2023b) Empagliflozin Improves the MicroRNA Signature of Endothelial Dysfunction in Patients with Heart Failure with Preserved Ejection Fraction and Diabetes. J Pharmacol Exp Ther 384:116-122.
78. Santulli G, Morelli M B and Gambardella J (2020) Is endothelial dysfunction the concealed cornerstone of COVID-19? BMJ 368:m1091.
79. Gambardella J, Jankauskas S S, D'Ascia S L, Sardu C, Matarese A, Minicucci F, Mone P and Santulli G (2022) Glycation of ryanodine receptor in circulating lymphocytes predicts the response to cardiac resynchronization therapy. J Heart Lung Transplant 41:438-441.
80. Gambardella J and Santulli G (2021) What is linking COVID-19 and endothelial dysfunction? Updates on nanomedicine and bioengineering from the 2020 AHA Scientific Sessions. Eur Heart J Cardiovasc Pharmacother 7:e2-e3.
81. Mone P, Gambardella J, Wang X, Jankauskas S S, Matarese A and Santulli G (2021) miR-24 Targets the Transmembrane Glycoprotein Neuropilin-1 in Human Brain Microvascular Endothelial Cells. Noncoding RNA 7.
82. Kansakar U, Gambardella J, Varzideh F, Avvisato R, Jankauskas S S, Mone P, Matarese A and Santulli G (2022) miR-142 Targets TIM-1 in Human Endothelial Cells: Potential Implications for Stroke, COVID-19, Zika, Ebola, Dengue, and Other Viral Infections. Int J Mol Sci 23.
83. Gambardella J, Kansakar U, Sardu C, Messina V, Jankauskas S S, Marfella R, Maggi P, Wang X, Mone P, Paolisso G, Sorriento D and Santulli G (2023) Exosomal miR-145 and miR-885 Regulate Thrombosis in COVID-19. J Pharmacol Exp Ther 384:109-115.
84. Xie Y and Al-Aly Z (2022) Risks and burdens of incident diabetes in long COVID: a cohort study. Lancet Diabetes Endocrinol 10:311-321.
85. Qeadan F, Tingey B, Egbert J, Pezzolesi M G, Burge M R, Peterson K A and Honda T (2022) The associations between COVID-19 diagnosis, type 1 diabetes, and the risk of diabetic ketoacidosis: A nationwide cohort from the U S using the Cerner Real-World Data. PLOS One 17:e0266809.
86. Schmitt J A, Ashraf A P, Becker D J and Sen B (2022) Changes in Type 2 diabetes trends in Children and Adolescents during the COVID-19 Pandemic. J Clin Endocrinol Metab.
87. Alfayez O M, Aldmasi K S, Alruwais N H, Bin Awad N M, Al Yami M S, Almohammed O A and Almutairi A R (2022) Incidence of Diabetic Ketoacidosis Among Pediatrics With Type 1 Diabetes Prior to and During COVID-19 Pandemic: A Meta-Analysisof Observational Studies. Front Endocrinol (Lausanne) 13:856958.
88. Cha S, Seo E H, Lee S H, Kim K S, Oh C S, Moon J S and Kim J K (2021) MicroRNA Expression in Extracellular Vesicles from Nasal Lavage Fluid in Chronic Rhinosinusitis. Biomedicines 9.
89. Esmaeili A, Hosseini S and Baghaban Eslaminejad M (2021) Engineered-extracellular vesicles as an optimistic tool for microRNA delivery for osteoarthritis treatment. Cell Mol Life Sci 78:79-91.
90. Gu C, Zhang H and Gao Y (2021) Adipose mesenchymal stem cells-secreted extracellular vesicles containing microRNA-192 delays diabetic retinopathy by targeting ITGA1. J Cell Physiol 236:5036-5051.
91. Liao T L, Chen Y M, Tang K T, Chen P K, Liu H J and Chen D Y (2021) MicroRNA-223 inhibits neutrophil extracellular traps formation through regulating calcium influx and small extracellular vesicles transmission. Sci Rep 11:15676.
92. Liu Y, Li Y P, Xiao L M, Chen L K, Zheng S Y, Zeng E M and Xu C H (2021) Extracellular vesicles derived from M2 microglia reduce ischemic brain injury through microRNA-135a-5p/TXNIP/NLRP3 axis. Lab Invest 101:837-850.
93. Papiewska-Pajak I, Przygodzka P, Krzyzanowski D, Soboska K, Szulc-Kielbik I, Stasikowska-Kanicka O, Boncela J, Wagrowska-Danilewicz M and Kowalska M A (2021) Snail Overexpression Alters the microRNA Content of Extracellular Vesicles Released from HT29 Colorectal Cancer Cells and Activates Pro-Inflammatory State In Vivo. Cancers (Basel) 13.
94. Silvestro S, Gugliandolo A, Chiricosta L, Diomede F, Trubiani O, Bramanti P, Pizzicannella J and Mazzon E (2021) MicroRNA Profiling of HL-1 Cardiac Cells-Derived Extracellular Vesicles. Cells 10.
95. Sonoda Y, Kano F and Murata M (2021) Applications of cell resealing to reconstitute microRNA loading to extracellular vesicles. Sci Rep 11:2900.
96. Wang L, Wang Y, Xiang Y, Ma J, Zhang H, Dai J, Hou Y, Yang Y, Ma J and Li H (2021) An In Vitro Study on Extracellular Vesicles From Adipose-Derived Mesenchymal Stem Cells in Protecting Stress Urinary Incontinence Through MicroRNA-93/F3 Axis. Front Endocrinol (Lausanne) 12:693977.
97. Yang H C and Rhee W J (2021) Single Step In Situ Detection of Surface Protein and MicroRNA in Clustered Extracellular Vesicles Using Flow Cytometry. J Clin Med 10.
98. Chen W, Zhang F, Hou X, Xu H and Tang D (2022) Ameliorating role of microRNA-378 carried by umbilical cord mesenchymal stem cells-released extracellular vesicles in mesangial proliferative glomerulonephritis. Cell Commun Signal 20:28.
99. Fullerton J L, Cosgrove C C, Rooney R A and Work L M (2022) Extracellular vesicles and their microRNA cargo in ischaemic stroke. J Physiol.
100. Pavani K C, Meese T, Pascottini O B, Guan X, Lin X, Peelman L, Hamacher J, Van Nieuwerburgh F, Deforce D, Boel A, Heindryckx B, Tilleman K, Van Soom A, Gadella B M, Hendrix A and Smits K (2022) Hatching is modulated by microRNA-378a-3p derived from extracellular vesicles secreted by blastocysts. Proc Natl Acad Sci USA 119:e2122708119.
101. Huang C W, Yu A S, Song H, Park J S, Wu S S, Khang V K, Subject C C and Shen E (2022) Association Between Dexamethasone Treatment After Hospital Discharge for Patients With COVID-19 Infection and Rates of Hospital Readmission and Mortality. JAMA Netw Open 5:e221455.
102. Santulli G, Pascale V, Finelli R, Visco V, Giannotti R, Massari A, Morisco C, Ciccarelli M, Illario M, Iaccarino G and Coscioni E (2019) We are What We Eat: Impact of Food from Short Supply Chain on Metabolic Syndrome. J Clin Med 8:2061.
103. Fader K A, Zhang J, Menetski J P, Thadhani R I, Antman E M, Friedman G S, Ramaiah S K and Vaidya V S (2021) A Biomarker-Centric Approach to Drug Discovery and Development: Lessons Learned from the Coronavirus Disease 2019 Pandemic. J Pharmacol Exp Ther 376:12-20.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A method of identifying a COVID-19-positive subject at risk for developing diabetes, the method comprising:
(a) determining the level of a microRNA (miRNA) in a sample from the COVID-19-positive subject; and
(b) comparing the level of the miRNA to a control,
wherein a significantly higher level of the miRNA relative to the control indicates that the COVID-19-positive subject is at risk for developing diabetes, and wherein the sample comprises an endothelial cell extracellular vesicle (EC-EV).

2. The method of claim 1, wherein said significantly higher level of the miRNA comprises an at least 20% increase in the level of the miRNA.

3. The method of claim 1, wherein said significantly higher level of the miRNA comprises at least 50% increase in the level of the miRNA.

4. The method of claim 1, further comprising recommending, prescribing, and/or administering a therapy to prevent or treat diabetes to the COVID-19-positive subject determined to be at risk for developing diabetes.

5. A method of preventing or treating diabetes in a COVID-19-positive subject, the method comprising
(a) identifying a COVID-19-positive subject at risk for developing diabetes according to the method of claim 1; and
(b) administering to the subject a therapy to prevent or treat diabetes.

6. The method of claim 4, wherein the therapy to prevent or treat diabetes comprises insulin, biguanides, thiazolidinediones, Lyn kinase activator, secretagogues, alpha-glucosidase inhibitor, peptide analog, glycosurics, or any combination thereof.

7. The method of claim 1, further comprising administering a COVID-19 therapy to the subject.

8. The method of claim 7, wherein the COVID-19 therapy comprises remdesivir, PF-07321332, molnupiravir (Lagevrio), MitoQ, a cell-derived therapeutic exosome, berzosertib, Favipiravir, lopinavir/ritonavir with or without IFN-beta-1a, ASC-09 and ritonavir, CD24Fc, Bamlanivimab and/oretesevimab, Bebtelovimab, Casirivimab/imdevimab (REGEN-COV, Ronapreve), Regdanvimab (Regkirona), Sotrovimab, Tixagevimab AZD8895) and/or cilgavimab (AZD1061) (collectively called Evusheld), Nirmatrelvir/ritonavir (Paxlovid), baricitinib, ensovibep, convalescent plasma, tocilizumab (Actemra), lenzilumab, Dapagliflozin, Apabetalone, Sarilumab, Sabizabulin, or any combination thereof.

9. The method of claim 1, wherein the control comprises a level of the miRNA in
(a) a sample from a healthy subject, a COVID-19-negative subject, a subject without diabetes, or a COVID-19-positive subject without diabetes; or
(b) a portion or all of pooled samples from one or more subjects of (a).

10. The method of claim 1, wherein the control is a pre-determined level of the miRNA.

11. The method of claim 1, wherein the sample comprises body fluid of the subject, optionally plasma of the subject.

12. The method of claim 1, wherein the sample comprises a CD31-positive extracellular vesicle.

13. The method of claim 1, further comprising fractionating EVs from the sample before detecting the level of the miRNA.

14. The method of claim 1, further comprising isolating the endothelial cell extracellular vesicle (EC-EVs) and/or a CD31-positive extracellular vesicle from the sample before detecting the level of the miRNA.

15. The method of claim 1, further comprising reverse transcribing the miRNA into a cDNA before detecting the level of the miRNA.

16. The method of claim 1, wherein the level of the miRNA is detected by a method comprising: multiplex bead-based assays, RNA-seq, next generation sequencing, sequencing, mass spectrometry (e.g., RNA sequencing by LC-MS, cDNA sequencing by LC-MS), microarray, Southern blotting of the cDNA of miRNA, Northern blotting, PCR, RT-PCR, real-time PCR (e.g., TaqMan®), any variation thereof, or any combination of two or more thereof.

17. The method of claim 1, wherein the miRNA is miR-34a.

18. The method of claim 1, wherein the COVID-19-positive subject has long COVID.

19. The method of claim 1, wherein the subject is a mammal.

20. The method of claim 1, wherein the subject is a dog, a cat, or a human, optionally a human.


SEQUENCE LISTING (1 sequence)

SEQ ID NO: 1 - RNA, 110 nt, Homo sapiens
  source 1..110: mol_type = other RNA
  modified_base 8: mod_base = OTHER; note = thymine
  modified_base 10: mod_base = OTHER; note = thymine
  modified_base 14: mod_base = OTHER; note = thymine
  modified_base 16..18: mod_base = OTHER; note = thymine
  modified_base 20..22: mod_base = OTHER; note = thymine
  modified_base 28: mod_base = OTHER; note = thymine
  modified_base 30: mod_base = OTHER; note = thymine
  modified_base 32..33: mod_base = OTHER; note = thymine
  modified_base 37: mod_base = OTHER; note = thymine
  modified_base 40..41: mod_base = OTHER; note = thymine
  modified_base 43..44: mod_base = OTHER; note = thymine
  modified_base 46: mod_base = OTHER; note = thymine
  modified_base 53: mod_base = OTHER; note = thymine
  modified_base 56: mod_base = OTHER; note = thymine
  modified_base 67: mod_base = OTHER; note = thymine
  modified_base 75: mod_base = OTHER; note = thymine
  modified_base 77: mod_base = OTHER; note = thymine
  modified_base 80: mod_base = OTHER; note = thymine
  modified_base 85: mod_base = OTHER; note = thymine
  modified_base 91: mod_base = OTHER; note = thymine
  modified_base 94: mod_base = OTHER; note = thymine
  modified_base 100..101: mod_base = OTHER; note = thymine
  modified_base 103: mod_base = OTHER; note = thymine
  misc_feature 1..110: note = Homo sapiens microRNA 34a (MIR34a) (NCBI NR_029610.1)
ggccagctgtgagtgtttctttggcagtgtcttagctggttgttgtgagcaatagtaaggaagcaatcagcaagtatactgccctagaagtgctgcacgttgtggggccc